(19) United States
(12) Patent Application Publication
(10) Pub. No.: US 2025/0340861 A1
(43) Pub. Date: Nov. 6, 2025

(54) DISPLAY TECHNOLOGY LIBRA-SEQ AND METHODS OF USE THEREOF

(71) Applicant: VANDERBILT UNIVERSITY, Nashville, TN (US)

(72) Inventors: Hema Preethi Subas Satish, Nashville, TN (US); Ivelin Georgiev, Nashville, TN (US); Kelsey Pilewski, Nashville, TN (US)

(21) Appl. No.: 19/197,185

(22) Filed: May 2, 2025

Related U.S. Application Data
(60) Provisional application No. 63/641,673, filed on May 2, 2024.

(51) Int. Cl. C12N 15/10; C12N 15/81; C12Q 1/686; C12Q 1/6869

(52) U.S. Cl. CPC C12N 15/1037; C12N 15/1096; C12N 15/81; C12Q 1/686; C12Q 1/6869

(57) ABSTRACT

The present disclosure relates to methods for simultaneous detection of antigens and antibodies or host receptor proteins that specifically bind said antigens.

### CROSS REFERENCE TO RELATED APPLICATION

This application claims priority to and the benefit of U.S. Provisional Application No. 63/641,673, filed on May 2, 2024, the contents of which are hereby incorporated by reference in their entirety.

### STATEMENT REGARDING FEDERALLY SPONSORED RESEARCH

This invention was made with government support under Grant No. R01 AI152693 and Grant No. R01 AI175245 awarded by the National Institutes of Health. The government has certain rights in the invention.

### REFERENCE TO SEQUENCE LISTING

The sequence listing submitted on May 23, 2025, as an.XML file entitled “10644-166US1.xml” created on Apr. 30, 2025, and having a file size of 42,682 bytes in is hereby incorporated by reference pursuant to 37 C.F.R. § 1.835(a)(2).

### FIELD

The present disclosure relates to methods for simultaneous detection of antigens and antigen specific antibodies or host receptor proteins thereof.

### BACKGROUND

The human immune system participates in complex interactions with virtually all other systems in the body. In particular, the B-cell component of the adaptive immune response plays a role in various disease settings, including infectious disease, cancer, autoimmunity, cardiovascular, hematologic, and neurologic diseases. In addition, antibodies (a product of B cells) are effectively used in diagnostics and therapeutics.

Despite decades of progress in antibody discovery and sequencing technologies, efforts to comprehensively link antibody sequence to antigen specificity remain constrained by fundamental methodological limitations. Traditional approaches often decouple sequence acquisition from antigen-binding assays, resulting in workflows that are fragmented, labor-intensive, and inherently low-throughput. Even with next-generation sequencing (NGS) tools enabling rapid profiling of B-cell receptor (BCR) repertoires, the challenge of pairing these sequences with their corresponding antigens persists.

Existing methods for identifying potent antibodies such as single B-cell culture, hybridoma screening, or antigen-specific sorting are typically limited by throughput, antigen coverage, or the need for time-consuming cloning and expression steps. These constraints hinder the ability to detect rare clones, explore cross-reactivity, or interrogate diverse antigen panels in a scalable and integrated manner. Notably, conventional techniques rarely permit concurrent recovery of antibody sequence and functional specificity from the same cell.

These limitations underscore the need for improved methodologies that can efficiently and simultaneously resolve antibody-antigen or receptor-ligand relationships across complex immune repertoires. In addition, there is an unmet need for methodologies that can concurrently identify immune receptor sequences and determine their antigen specificity in a high-throughput, scalable, and precise manner. There remains a pressing need for integrated platforms that overcome the throughput bottlenecks of traditional techniques, permit multiplexed antigen screening, and enable comprehensive mapping of immune specificity at single-cell resolution, without reliance on laborious cloning or low-throughput expression systems.

### SUMMARY

Disclosed herein are methods for simultaneous detection of antigens and the antigen specific antibodies or host receptors thereof (also referred to herein as “Display Technology LInking B cell Receptor to Antigen specificity through Sequencing” or “dtLIBRA-Seq”).

Various aspects include a method for simultaneous detection of an antigen and an antibody thereof that specifically binds said antigen.

In some aspects, the method includes: constructing a cell-free barcoded antigen display library comprising a plurality of plasmids encoding a plurality of antigens and a plurality of antigen barcodes, wherein each plasmid comprises a nucleic acid sequence encoding an antigen and a unique antigen barcode; generating an antigen-barcode dictionary by mapping each unique antigen barcode to its corresponding antigen; performing in vitro transcription of each plasmid to produce an mRNA transcript, wherein the mRNA transcript encodes the antigen and the unique antigen barcode; reverse transcribing the mRNA transcript encoding the unique antigen barcode to form a corresponding cDNA; performing in vitro translation of the mRNA transcript to express a cell-free barcoded antigen; allowing a plurality of cell-free barcoded antigens to bind to a population of B-cells; washing unbound cell-free barcoded antigens from the population of B-cells; separating the B-cells bound to the cell-free barcoded antigens into single cell emulsions; introducing a unique cell barcode-labeled bead into each single cell emulsion; preparing a single cell cDNA library from each single cell emulsion, wherein the cDNA library comprises nucleic acid sequences encoding immunoglobulin heavy chain and/or immunoglobulin light chain sequences and the unique antigen barcode; performing PCR amplification reactions to generate a plurality of amplicons comprising: (1) the unique cell barcode, a unique molecular identifier (UMI) and the unique antigen barcode, (2) the unique cell barcode, the immunoglobulin heavy chain and/or immunoglobulin light chain sequences, and the unique molecular identifier (UMI); sequencing the plurality of amplicons and removing sequences lacking any of the unique cell barcode, the UMI, the unique antigen barcode, or the immunoglobulin sequence; aligning the immunoglobulin sequences to a reference library of V, D, J, and C gene segments to annotate antibody sequences; constructing a UMI count matrix comprising the unique cell barcode, the unique antigen barcode, and the corresponding antibody sequence; and determining a LIBRA-seq score for each antigen-antibody pair based on the UMI count matrix. In some aspects, the amplicons are tagged with unique molecular identifiers (UMIs) to quantify original transcript abundance and calculate antigen-binding scores.

In some aspects, the plasmid comprises a unique antigen barcode, a T7 promoter, a ribosome binding site (RBS), an N-terminal Tag, an epitope tag, and an antigen sequence. In some aspects, the N-terminal Tag comprises HaloTag, HA Tag or HIS Tag. In some aspects, the epitope tag is a FLAG tag, wherein the B-cells bound to the cell-free barcoded antigens are isolated using an antibody against the epitope tag.

In some aspects, the unique antigen barcode is reverse transcribed using a primer comprising a HaloLigand moiety, wherein the HaloLigand moiety covalently links to N-terminal HaloTag of translated antigens.

In some aspects, the cell-free barcoded antigens are not purified prior to incubation with the population of B-cells.

In some aspects, the unique antigen barcode comprises a degenerate at least 10-nucleotide long sequence synthesized from a randomized oligonucleotide pool.

In some aspects, the cell-free barcoded antigens comprise an antigen from a pathogen or an animal. In some aspects, the antigen from the animal comprises a tumor-associated antigen or a neoantigen. In some aspects, the antigen from a pathogen comprises an antigen from a nosocomial infection causing bacteria. In some aspects, the nosocomial infection causing bacteria comprises Staphylococcus aureus, Acinetobacter baumannii, Clostridioides difficile, or a combination thereof. In some aspects, the antigen from a pathogen comprises an antigen from a virus. In some aspects, the virus comprises HIV-1, SARS-CoV-2, SARS-CoV-1 or MERS.

In one aspect, disclosed herein is a library of barcode-labeled antigen proteins, comprising: a plurality of barcode-labeled antigen proteins, wherein each barcode-labeled antigen protein comprises: (i) a HaloTag, (ii) an epitope tag, wherein the epitope tag is a FLAG tag; (iii) an antigen protein and (iv) a unique antigen barcode, wherein the unique antigen barcode is covalently attached to the HaloTag of the antigen protein via a HaloLigand moiety.

In one aspect, disclosed herein is a method for simultaneous detection of an antigen and a host receptor protein that specifically binds said antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a protein-barcode dictionary by mapping each unique receptor protein barcode to its corresponding host receptor protein;
performing in vitro transcription of each plasmid to produce an mRNA transcript; wherein the mRNA transcript encodes the host receptor protein and the unique receptor protein barcode;
reverse transcribing the mRNA transcript encoding the unique receptor protein barcode to form a corresponding cDNA;
performing in vitro translation of the mRNA transcript to express a cell-free barcoded host receptor protein;
contacting a plurality of cell-free barcoded host receptor proteins with a yeast display library expressing a plurality of antigens, wherein each antigen is attached to a unique antigen barcode;
allowing the plurality of cell-free barcoded host receptor proteins to bind to the plurality of antigens of the yeast display library to form a receptor-antigen binding complex comprising cell-free barcoded host receptor proteins bound to yeast cells;
washing unbound cell-free barcoded host receptor proteins from antigen expressing yeast cells;
separating the antigen expressing yeast cells bound to the cell-free barcoded host receptor proteins into single cell emulsions;
introducing a unique cell barcode-labeled bead into each single cell emulsion;
preparing a single cell cDNA library from each single cell emulsion, wherein the cDNA library comprises nucleic acid sequences encoding the unique antigen barcode and the unique receptor protein barcode;
performing PCR amplification reactions to generate a plurality of amplicons comprising: (1) the unique cell barcode, a unique molecular identifier (UMI) and the unique antigen barcode, and (2) the unique cell barcode, the unique receptor protein barcode, and the unique molecular identifier (UMI);
sequencing the plurality of amplicons and removing sequences lacking any of the unique cell barcode, the UMI, the unique antigen barcode, or the unique receptor protein barcode;
constructing a UMI count matrix comprising the unique cell barcode, the unique receptor protein barcode, and the unique antigen barcode; and
determining a LIBRA-seq score for each antigen-receptor pair based on the UMI count matrix.

In some aspects, the yeast display library is prepared by the following method: preparing a plurality of yeast display vectors encoding a plurality of antigens, wherein each yeast display vector comprises a nucleic acid sequence for an antigen and a unique antigen barcode; generating an antigen-barcode dictionary by mapping each unique antigen barcode to its corresponding antigen; and transforming the yeast display vectors into Saccharomyces cerevisiae cells, wherein the S. cerevisiae cells induce surface expression of the yeast display vectors, thereby obtaining the yeast display library expressing the plurality of antigens with the unique antigen barcodes.

In some aspects, disclosed herein is a method for simultaneous detection of a host receptor protein and a neutralizing antibody that blocks the interaction of said host receptor protein with an antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a receptor protein-barcode dictionary by mapping each unique receptor protein barcode to its corresponding host receptor protein;
expressing the plurality of plasmids in a cell-free system; thereby obtaining barcoded host receptor proteins;
constructing a barcoded antigen library comprising a plurality of plasmids encoding a plurality of antigens;
expressing the plurality of antigens in a cell culture; wherein each antigen is associated with a unique antigen barcode, thereby obtaining barcoded antigens;
contacting B cells to the barcoded antigens and a barcoded host receptor protein-barcoded antigen complex;
sorting B cells bound to the barcoded antigens and/or the barcoded host receptor protein-barcoded antigen complex;
partitioning the sorted B cells into single-cell emulsions;
introducing a unique cell barcode-labeled bead into each single-cell emulsion;
synthesizing cDNA from each sorted B cell;
amplifying immunoglobulin variable region sequences (V(D)J) sequences, from each B cell with the unique antigen barcode and the unique receptor protein barcode, wherein the V(D)J sequences encode the antigen-binding regions of B cell receptors (BCRs) or secreted antibodies;
performing PCR amplification reactions to generate a plurality of amplicons comprising: (1) the unique cell barcode, a unique molecular identifier (UMI), and the unique antigen barcode, and (2) the unique cell barcode, the unique receptor protein barcode, and the unique molecular identifier (UMI);
sequencing the plurality of amplicons and removing sequences lacking any of the unique cell barcode, the UMI, the unique antigen barcode, or the unique receptor protein barcode;
constructing a UMI count matrix comprising the unique cell barcode, the unique receptor protein barcode, and the unique antigen barcode;
calculating a LIBRA-seq score for each combination comprising the unique cell barcode, the unique receptor protein barcode, and the unique antigen barcode based on the UMI count matrix; and
identifying the neutralizing antibody that blocks the interaction of the host receptor protein with an antigen.

In some aspects, the cell-free barcoded host receptor proteins comprise a receptor protein associated with viral infection. In some aspects, the receptor proteins comprise human receptor proteins. In some aspects, the human receptor proteins comprise proteins from human epithelial cells.

### BRIEF DESCRIPTION OF DRAWINGS

The accompanying figures, which are incorporated in and constitute a part of this specification, illustrate aspects described below.

FIGS. 1A, 1B and 1C show a schematic of open reading frame generation and cloning. FIG. 1A show generation of ORFs for gateway cloning into entry vector. FIG. 1B shows custom cell-free barcoded antigen/protein display plasmid library. FIG. 1C shows Gateway cloning™, which resembles the in vivo integration and excision recombination reactions that occurs when lambda phage infects bacteria.

FIG. 2 shows the generation of cDNA barcode-antigen/host protein complex.

FIG. 3 shows high-throughput simultaneous identification of antigen-antibody pairs by screening for antigen-specific B cells using cell-free barcoded antigen display library.

FIGS. 4A and 4B show an overview of high-throughput simultaneous identification of antigens and associated receptors. FIG. 4A shows high-throughput simultaneous identification of host (human) receptor/protein associated with pathogen invasion/infection by screening cell-free human receptor/protein display library using antigen yeast display library. FIG. 4B shows high-throughput simultaneous identification of host (human) receptors and potently neutralizing antibodies against the pathogen via ligand blocking.

### DETAILED DESCRIPTION

Disclosed herein are systems and methods for simultaneous detection of antigens and antigens specific to antibodies or binding fragments thereof.

Reference will now be made in detail to the embodiments of the invention, examples of which are illustrated in the drawings and the examples. This invention may, however, be embodied in many different forms and should not be construed as limited to the embodiments set forth herein. Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an inter nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here can include the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

As used herein, the term “antigen” refers to a molecule that is capable of binding to an antibody. In some embodiments, the antigen stimulates an immune response such as by production of antibodies specific for the antigen. Antigens of the present disclosure can be, for example, an antigen from human immunodeficiency virus (HIV), an antigen from influenza virus, or an antigen from respiratory syncytial virus (RSV). Antigens of the present disclosure can also be, for example, a human antigen (e.g. VEGF, or an oncogene-encoded protein).

In the present disclosure, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to specifically interact with the HIV virus, such that the HIV viral infection is prevented, inhibited, reduced, or delayed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mice. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In humans, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. The term “Fab region”, as used herein, refers to the region of an antibody composed of one constant and one variable domain from each heavy and light chain of the antibody, and which contains the sites involved in antigen binding. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, the term “antigen-specific B-cell” refers to a B-cell that expresses antibodies that are able to distinguish between an antigen of interest and other antigens. The antigen specific B cell specifically bind to that antigen of interest with high or low affinity, but which do not bind to other antigens.

As used herein, the term “unique antigen barcode” refers to a nucleic acid sequence associated with a specific antigen that serves as a molecular identifier, enabling the correlation of antigen identity with downstream biological or analytical readouts. Non-limiting examples include short synthetic DNA sequences of about 10, 15, 20, 25, 30, 35, 40, 45 or 50 nucleotides designed to be distinct across different antigens in a library.

As used herein, the term “T7 promoter” refers to a DNA sequence recognized by T7 RNA polymerase, enabling transcription of downstream sequences in either in vitro or in vivo systems. Non-limiting examples of T7 promoters include the canonical T7 consensus sequence.

As used herein, the term “ribosome binding site (RBS)” refers to a sequence element that facilitates the binding of ribosomes to mRNA, thereby promoting translation initiation. In some aspects, the RBS may comprise a Shine-Dalgarno sequence positioned upstream of the start codon.

As used herein, the term “N-terminal tag” refers to a proteinaceous or peptide tag fused to the N-terminus of a polypeptide/antigen to facilitate expression, detection, purification, or functionalization of the polypeptide. Non-limiting examples of N-terminal tags include: HaloTag which is a modified haloalkane dehalogenase that covalently binds to synthetic ligands; HA Tag (Hemagglutinin Tag) which is an epitope derived from influenza hemagglutinin protein, and His Tag (Poly histidine Tag) is a series of consecutive histidine residues, typically six (6xHis), facilitating metal-affinity purification. In some embodiments, a Spytag can be used (binds spycatcher). In some embodiments, a stop codon to incorporate unnatural amino acid (UAA) containing azide groups such as p-azido-L-phenylalanine can be used that can be conjugated with a DBCO-modified primer via copper-free click chemistry or other UAAs that allow simple but rapid reactions.

As used herein, the term “epitope tag” refers to a short, recognizable peptide sequence incorporated into a protein of interest to enable detection or affinity-based isolation using specific antibodies. Non-limiting examples include a FLAG Tag, a Myc Tag, or a V5 Tag. In some aspects, the epitope tag is a FLAG tag, wherein B-cells bound to cell-free barcoded antigens are isolated using an anti-FLAG antibody (e.g., M2 monoclonal antibody). In some embodiments, HA Tag (Hemagglutinin Tag) which is an epitope derived from influenza hemagglutinin protein, and His Tag (Poly histidine Tag) which is a series of consecutive histidine residues, typically six (6xHis), facilitating metal-affinity purification, can be used as an epitope tag with HaloTag as the N-terminal tag.

As used herein, the term “cell-free barcoded antigen” refers to an antigen protein that has been expressed in a cell-free system from a DNA template comprising an antigen-encoding sequence linked to a unique oligonucleotide barcode. The resulting antigens may be detected, tracked, or isolated using the barcode or associated tags (e.g., HaloTag, FLAG tag). As used herein, the term “unique antigen barcode” refers to a distinct nucleic acid sequence incorporated into each plasmid or its transcript, allowing individual identification of each encoded antigen.

As used herein, the term “in vitro transcription” refers to the enzymatic synthesis of RNA from a DNA template outside of living cells, typically using T7 RNA polymerase or equivalent enzymes.

As used herein, the term “in vitro translation” refers to the process of synthesizing proteins from an mRNA template using cell-free expression systems, such as bacterial lysates, wheat germ extracts, or other reconstituted systems.

As used herein, the term “single cell cDNA library” refers to a collection of nucleic acid sequences prepared from individual cells, comprising immunoglobulin heavy and/or light chain sequences and antigen barcodes, each tagged with a unique cell barcode.

As used herein, the term “unique molecular identifier (UMI)” refers to a short, random nucleic acid sequence included in nucleic acid libraries to uniquely tag individual molecules and distinguish original molecules from amplification artifacts.

As used herein, the term “reference library of V, D, J, and C gene segments” refers to curated databases containing known immunoglobulin variable (V), diversity (D), joining (J), and constant (C) region gene sequences, used for annotating antibody sequences.

As used herein, the term “UMI count matrix” refers to a data structure that tabulates the relationships among unique cell barcodes, unique antigen barcodes, corresponding antibody sequences, and associated UMI counts.

As used herein, the term “LIBRA-seq score” refers to a numerical value derived from the UMI count matrix representing the strength or confidence of the association between a particular antigen and a corresponding antibody.

### Methods

Disclosed herein are methods for simultaneous detection of an antigen and an antibody (or host receptor protein) that specifically binds said antigen.

In some aspects, disclosed herein is a method for simultaneous detection of an antigen and an antibody that specifically binds said antigen, comprising:
constructing a cell-free barcoded antigen display library comprising a plurality of plasmids encoding a plurality of antigens and a plurality of antigen barcodes, wherein each plasmid comprises a nucleic acid sequence encoding an antigen and a unique antigen barcode;
generating an antigen-barcode dictionary by mapping each unique antigen barcode to its corresponding antigen;
performing in vitro transcription of each plasmid to produce an mRNA transcript, wherein the mRNA transcript encodes the antigen and the unique antigen barcode;
reverse transcribing the mRNA transcript encoding the unique antigen barcode to form a corresponding cDNA;
performing in vitro translation of the mRNA transcript to express a cell-free barcoded antigen;
allowing a plurality of cell-free barcoded antigens to bind to a population of B-cells;
washing unbound cell-free barcoded antigens from the population of B-cells;
separating the B-cells bound to the cell-free barcoded antigens into single cell emulsions;
introducing a unique cell barcode-labeled bead into each single cell emulsion;
preparing a single cell cDNA library from each single cell emulsion, wherein the cDNA library comprises nucleic acid sequences encoding immunoglobulin heavy chain and/or immunoglobulin light chain sequences and the unique antigen barcode;
performing PCR amplification reactions to generate a plurality of amplicons comprising: (1) the unique cell barcode, a unique molecular identifier (UMI) and the unique antigen barcode, (2) the unique cell barcode, the immunoglobulin heavy chain and/or immunoglobulin light chain sequences, and the unique molecular identifier (UMI);
sequencing the plurality of amplicons and removing sequences lacking any of the unique cell barcode, the UMI, the unique antigen barcode, or the immunoglobulin sequence;
aligning the immunoglobulin sequences to a reference library of V, D, J, and C gene segments to annotate antibody sequences;
constructing a UMI count matrix comprising the unique cell barcode, the unique antigen barcode, and the corresponding antibody sequence; and
determining a LIBRA-seq score for each antigen-antibody pair based on the UMI count matrix.

In some aspects, disclosed herein is a method for simultaneous detection of an antigen and an antibody that specifically binds said antigen, comprising:
constructing a cell-free barcoded antigen display library comprising a plurality of plasmids encoding a plurality of antigens and a plurality of antigen barcodes, wherein each plasmid comprises a nucleic acid sequence encoding an antigen and a unique antigen barcode;
generating an antigen-barcode dictionary by mapping each unique antigen barcode to its corresponding antigen;
performing in vitro transcription of each plasmid to produce an mRNA transcript, wherein the mRNA transcript encodes the antigen and the unique antigen barcode;
reverse transcribing the mRNA transcript encoding the unique antigen barcode to form a corresponding cDNA;
performing in vitro translation of the mRNA transcript to express a cell-free barcoded antigen;
allowing a plurality of cell-free barcoded antigens to bind to a population of B-cells;
separating the B-cells bound to the cell-free barcoded antigens into single cell emulsions;
introducing a unique cell barcode-labeled bead into each single cell emulsion;
preparing a single cell cDNA library from each single cell emulsion, wherein the cDNA library comprises nucleic acid sequences encoding immunoglobulin heavy chain and/or immunoglobulin light chain sequences and the unique antigen barcode;
performing PCR amplification reactions to generate a plurality of amplicons comprising: (1) the unique cell barcode, a unique molecular identifier (UMI) and the unique antigen barcode, (2) the unique cell barcode, the immunoglobulin heavy chain and/or immunoglobulin light chain sequences, and the unique molecular identifier (UMI); and
sequencing the plurality of amplicons.

As used herein, the term “unique antigen barcode” refers to a distinct nucleic acid sequence associated with a nucleic acid encoding an antigen, wherein the barcode serves as a molecular identifier uniquely corresponding to the antigen in a LIBRA-seq workflow. During LIBRA-seq, the unique antigen barcode enables the identification and tracking of antigen-specific B-cell interactions by linking sequencing information to specific antigens. In some aspects, the unique antigen barcode is incorporated into the expressed construct such that it is captured and sequenced together with the B-cell receptor (BCR) transcripts, allowing determination of antigen specificity at the single-cell level.

In some aspects, the plasmid comprises a unique antigen barcode, a T7 promoter, a ribosome binding site (RBS), an N-terminal Tag, an epitope tag, and an antigen sequence. In some aspects, the N-terminal Tag comprises HaloTag, HA Tag, or HIS Tag. In some aspects, the epitope tag is a FLAG tag, wherein the B-cells bound to the cell-free barcoded antigens are isolated using an antibody against the epitope tag.

In some aspects, the unique antigen barcode is reverse transcribed using a primer comprising a HaloLigand moiety, wherein the HaloLigand moiety covalently links to N-terminal HaloTag of the translated antigens. In some aspects, antigens or host receptors are expressed as fusion proteins with HaloTag to facilitate stable and specific conjugation to DNA barcodes. The HaloTag-fused antigens are covalently labeled using synthetic HaloTag ligands that are pre-functionalized with nucleic acid barcodes. This approach enables the formation of a stable and irreversible linkage between each antigen and its corresponding barcode, which is particularly advantageous in high-throughput screening applications such as dtLIBRA-seq. The covalent nature of the HaloTag-ligand interaction ensures that the barcode remains permanently associated with the antigen throughout various downstream processes, including rigorous washing and cell sorting. Furthermore, this system is well-suited for use with cell-free protein expression platforms, which are employed in dtLIBRA-seq workflows to enable rapid and parallel antigen production.

In some embodiments, a cell-free barcoded antigen display library is generated for high-throughput antigen screening applications such as dtLIBRA-seq. The process begins with the construction of a plasmid library encoding a diverse set of antigens/receptor proteins/full-length proteins, each linked to a unique nucleic acid barcode.

As used herein, the term “cell-free barcoded antigen display library” refers to a collection of expression constructs, each encoding a unique antigen (e.g., peptide or full-length protein) that is physically linked to a unique nucleic acid barcode, and wherein protein expression is performed in a cell-free system. This system enables parallel and scalable in vitro expression of a highly diverse antigen repertoire, each antigen bearing a distinct barcode sequence that permits subsequent identification and correlation with immune receptor sequences (e.g., B cell receptor or antibody sequences).

In certain embodiments, the expression vector further comprises an integrated barcode cassette containing a degenerate oligonucleotide pool representing a diverse set of unique 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45-nucleotide barcode sequences. These barcode cassettes are cloned into the destination vector, thereby eliminating the need for a separate barcode conjugation step and enhancing barcode diversity and protein recovery. The use of a degenerate oligo pool enables the generation of a barcode repertoire with theoretical complexity exceeding 10⁹ to 10¹⁰ distinct sequences.

Following construction of the final barcoded expression constructs, the plasmid library is transformed into electrocompetent E. coli cells (e.g., ElectroMAX™ DH10B) for amplification, yielding a diverse population of plasmids containing unique antigen-barcode combinations. The amplified plasmid library is subsequently used as a template in a cell-free transcription and translation system (e.g., T7-based lysate systems) to express a barcoded antigen library in vitro.

The resulting barcoded antigen display library can be used for a wide range of applications, including identification of antibody-antigen binding interactions, discovery of tumor-associated antigens and neoantigens, immune repertoire mapping, and high-throughput serological profiling. In some embodiments, the antigen barcodes are captured along with single-cell antibody or receptor sequences using techniques such as droplet-based emulsion PCR or single-cell RNA-sequencing, enabling direct correlation of immune receptor specificity with antigen identity at the single-cell level.

In certain embodiments, the present disclosure provides methods for constructing an antigen-barcode dictionary using Oxford Nanopore Technologies (ONT) long-read sequencing. As used herein, the term “antigen-barcode dictionary” refers to a dataset or reference map that associates each antigen sequence encoded in a plasmid or expression construct with its corresponding unique nucleic acid barcode. The construction of this dictionary is used for downstream decoding and mapping of immune receptor specificity, particularly in high-throughput barcoded antigen screening platforms such as dtLIBRA-seq.

The antigen-barcode dictionary is generated by sequencing the full-length constructs from the plasmid library using ONT's nanopore-based long-read sequencing platform, which allows for the direct sequencing of continuous DNA molecules without fragmentation. In a preferred embodiment, sequencing is performed in the long-read mode, yielding read lengths ranging from approximately 10 kilobases (kb) to 100 kb. In alternative embodiments, ultra-long read sequencing may be employed to achieve read lengths from approximately 100 kb to 300 kb or greater, with the longest reads exceeding 4 megabases (Mb) in length. The use of long-read sequencing enables full coverage of the expression cassette including the antigen coding sequence, fusion tag elements (e.g., HaloTag), and the associated barcode sequence in a single contiguous read. This comprehensive read-through capability ensures unambiguous pairing of each antigen with its corresponding barcode, thereby overcoming limitations associated with short-read sequencing platforms that may fail to span both regions in a single read.

In some embodiments, the plasmid DNA is linearized or amplified via long-range PCR prior to library preparation to optimize sequencing performance and minimize concatemer artifacts. The resulting reads are aligned to a reference antigen and barcode library using high-accuracy base calling and mapping algorithms to generate a definitive lookup table linking each unique barcode to a specific antigen identity. This antigen-barcode dictionary may subsequently be used to decode barcode sequences recovered from antigen-bound B cells or other display platforms, enabling precise identification of antigen specificity in single-cell or bulk screening formats.

In some aspects, the cell-free barcoded antigens are not purified prior to incubation with the population of B-cells.

In some aspects, the unique antigen barcode comprises a degenerate at least 10-nucleotide long sequence synthesized from a randomized oligonucleotide pool.

It should be understood that the barcode described above is conjugated to the barcode-labeled antigen in a way that is known to one of ordinary skill in the art. Conjugates can be chemically linked to the nucleotide or nucleotide analogs.

As used herein, “beads” is not limited to a specific type of bead. Rather, a large number of beads are available and are known to one of ordinary skill in the art. A suitable bead may be selected on the basis of the desired end use and suitability for various protocols. In some embodiments, the barcode-labeled antigens are labeled with a first barcode comprising a DNA sequence or an RNA sequence. In some embodiments, the barcode-labeled antigens are labeled with a first barcode comprising a DNA sequence. In some embodiments, the barcode-labeled antigens are labeled with a first barcode comprising an RNA sequence.

In some aspects, the cell barcode-labeled beads are labeled with a second barcode comprising a DNA sequence or an RNA sequence. In some embodiments, the cell barcode-labeled beads are labeled with a second barcode comprising a DNA sequence. In some embodiments, the cell barcode-labeled beads are labeled with a second barcode comprising an RNA sequence. In some embodiments, the cell barcode-labeled beads are labeled with a barcode on the inside of the bead. In some embodiments, the cell barcode-labeled beads are labeled with a barcode encapsulated within the bead. In some embodiments, the cell barcode-labeled beads are labeled with a barcode on the outside of the bead.

These oligos, which contain the cell barcode, both: (1) enable amplification of cellular mRNA transcripts through the template switch oligo that is part of the oligo containing the cell barcode, and (2) directly anneal to the antigen barcode-containing oligos from the antigen. In some embodiments, the oligos delivered from the beads have the general structure: P5_PCR_handle-Cell_barcode-UMI-Template_switch_oligo.

In some aspects, the cell-free the cell-free barcoded antigens comprise an antigen from a pathogen or an animal. In some aspects, the animal is a mammal, including, but not limited to, primates (e.g., humans and nonhuman primates), cows, sheep, goats, horses, dogs, cats, rabbits, rats, mice and the like. In some aspects, the animal is a human. In some aspects, the antigen from the animal comprises an antigen from a tumor-associated antigen or neoantigen. “Neoantigens” as used herein generally include antigens that are present on the surface of cancer cells but are absent from the surface of normal cells of a patient. In some aspects, the antigen from a pathogen comprises an antigen from a nosocomial infection causing bacteria. In some aspects, the nosocomial infection causing bacteria comprises C. difficile, S. aureus, A. baumannii, or a combination thereof. In some aspects, the antigen from a pathogen comprises an antigen from a virus. In some aspects, the virus comprises HIV-1, SARS-CoV-2, SARS-CoV-1 or MERS.

In some aspects, the plurality of antigens comprises a panel of epitope knock-outs. In some aspects, the plurality of antigens comprises a panel of antigen variants or mutations for epitope mapping. For example, a mutant display library of antigens can be formed by introducing point mutations in residues of an antigen of interest before cloning said constructs into a mammalian surface display vector (e.g., via gateway cloning).

In some embodiments, the plurality of antigens or the plurality of host receptor proteins utilize donor vectors, such as pDONR222, pDONR221, pDONR/Zeo, or pDONR201, containing attP1 and attP2 recombination sites, serving as initial sites for capturing DNA sequences through BP clonase reactions. entry clone vector, designed for efficient cloning and recombination. The donor vectors serve as initial cloning sites for capturing DNA sequences of interest through BP clonase reactions, in which attB-flanked PCR products or DNA fragments recombine with the attP sites in the donor vector. This BP reaction yields an entry clone vector, which contains attL1 and attL2 recombination sites and carries the desired DNA fragment. Examples of suitable entry clone vectors include pENTR221, pENTR1A, pENTR/D-TOPO, and pENTR4 vectors. Additionally, the expression of the plurality of antigens or the plurality of host receptor proteins with the unique barcodes utilize destination vectors, characterized by recombination-compatible sites such as attR1 and attR2, enabling transfer of antigens or host receptor proteins sequences from entry clone vectors via recombination reactions. Destination vectors include but are not limited to pDEST17, pDEST15, pDEST10, pDEST14, pDEST26, or pDEST8 vectors.

In one aspect, disclosed herein is a library of barcode-labeled antigen proteins, comprising: a plurality of barcode-labeled antigen proteins, wherein each barcode-labeled antigen protein comprises: (i) a HaloTag, (ii) an epitope tag, wherein the epitope tag is a FLAG tag; (iii) an antigen protein and (iv) a unique antigen barcode, wherein the unique antigen barcode is covalently attached to the HaloTag of the antigen protein via a HaloLigand moiety.

A number of single cell workflows can be utilized in the present systems and methods for the simultaneous detection of antigens and antigen specific antibodies or binding fragment thereof. For example, the systems and methods can use microwell arrays or microwell cartridges (e.g., BD Rhapsody™) or microfluidics devices (e.g., 10x Genomics (San Francisco, Calif.), Drop-seq (McCarroll Lab, Harvard Medical School (Cambridge, Mass.); Macosko et al., Cell, 2015 May 21 16; 5:1202, the content of which is incorporated herein by reference in its entirety), or Abseq (Mission Bio (San Francisco, Calif.); Shahi et al., Sci Rep. 2017 Mar. 14; 7:44447, the content of which is hereby incorporated by reference in its entirety) in combination with solid or semisolid particles associated with barcodes, such as stochastic barcodes (e.g., BD Rhapsody, or Drop-seq), or disruptable hydrogel particles enclosing releasable barcodes, such as stochastic barcodes (e.g., 10x Genomics, or Abseq). Single cell partitions, such as, for example, microwell cartridges (e.g., BD Rhapsody™) comprising unique barcode sequences in each partition (e.g., well) can enable a user to associate cell labels from sequencing data with a particular partition. Examples suitable workflows include those discussed in U.S. Pat. App. Pub. No. 2021/0302422A1, which is hereby incorporated by reference in its entirety.

Also disclosed herein is a method for simultaneous detection of an antigen and a host receptor protein that specifically binds said antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a protein-barcode dictionary by mapping each unique receptor protein barcode to its corresponding host receptor protein;
performing in vitro transcription of each plasmid to produce an mRNA transcript; wherein the mRNA transcript encodes the host receptor protein and the unique receptor protein barcode;
reverse transcribing the mRNA transcript encoding the unique receptor protein barcode to form a corresponding cDNA;
performing in vitro translation of the mRNA transcript to express a cell-free barcoded host receptor protein;
contacting a plurality of cell-free barcoded host receptor proteins with a yeast display library expressing a plurality of antigens, wherein each antigen is attached to a unique antigen barcode;
allowing the plurality of cell-free barcoded host receptor proteins to bind to the plurality of antigens of the yeast display library to form a receptor-antigen binding complex comprising cell-free barcoded host receptor proteins bound to yeast cells;
washing unbound cell-free barcoded host receptor proteins from antigen expressing yeast cells;
separating the antigen expressing yeast cells bound to the cell-free barcoded host receptor proteins into single cell emulsions;
introducing a unique cell barcode-labeled bead into each single cell emulsion;
preparing a single cell cDNA library from each single cell emulsion, wherein the cDNA library comprises nucleic acid sequences encoding the unique antigen barcode and the unique receptor protein barcode;
performing PCR amplification reactions to generate a plurality of amplicons comprising: (1) the unique cell barcode, a unique molecular identifier (UMI) and the unique antigen barcode, and (2) the unique cell barcode, the unique receptor protein barcode, and the unique molecular identifier (UMI);
sequencing the plurality of amplicons and removing sequences lacking any of the unique cell barcode, the UMI, the unique antigen barcode, or the unique receptor protein
constructing a UMI count matrix comprising the unique cell barcode, the unique receptor protein barcode, and the unique antigen barcode; and
determining a LIBRA-seq score for each antigen-receptor pair based on the UMI count matrix.

Also disclosed herein is a method for simultaneous detection of an antigen and a host receptor protein that specifically binds said antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a protein-barcode dictionary by mapping each unique receptor protein barcode to its corresponding host receptor protein;
performing in vitro transcription of each plasmid to produce an mRNA transcript; wherein the mRNA transcript encodes the host receptor protein and the unique receptor protein barcode;
reverse transcribing the mRNA transcript encoding the unique receptor protein barcode to form a corresponding cDNA;
performing in vitro translation of the mRNA transcript to express a cell-free barcoded host receptor protein;
contacting a plurality of cell-free barcoded host receptor proteins with a yeast display library expressing a plurality of antigens, wherein each antigen is attached to a unique antigen barcode;
allowing the plurality of cell-free barcoded host receptor proteins to bind to the plurality of antigens of the yeast display library to form a receptor-antigen binding complex comprising cell-free barcoded host receptor proteins bound to yeast cells;
washing unbound cell-free barcoded host receptor proteins from antigen expressing yeast cells;
separating the antigen expressing yeast cells bound to the cell-free barcoded host receptor proteins into single cell emulsions;
introducing a unique cell barcode-labeled bead into each single cell emulsion; preparing a single cell cDNA library from each single cell emulsion, wherein the cDNA library comprises nucleic acid sequences encoding the unique antigen barcode and the unique receptor protein barcode;
performing PCR amplification reactions to generate a plurality of amplicons comprising: (1) the unique cell barcode, a unique molecular identifier (UMI) and the unique antigen barcode, and (2) the unique cell barcode, the unique receptor protein barcode, and the unique molecular identifier (UMI); and
sequencing the plurality of amplicons.

As used herein, the term “unique receptor protein barcode” refers to a distinct nucleic acid sequence operably linked to a nucleic acid encoding a receptor protein, serving as a molecular identifier uniquely corresponding to the receptor protein in a LIBRA-seq or dtLIBRA-seq. The unique receptor protein barcode enables high-throughput mapping of receptor-ligand interactions by allowing simultaneous sequencing of receptor identity and binding specificity in single-cell emulsions. In some aspects, the receptor barcode is captured alongside cellular transcript information, enabling direct association of receptor sequence data with bound antigens or ligands during downstream analysis.

In some aspects, the yeast display library is prepared by the following method:
preparing a plurality of yeast display vectors encoding a plurality of antigens, wherein each yeast display vector comprises a nucleic acid sequence for an antigen and a unique antigen barcode;
generating an antigen-barcode dictionary by mapping each unique antigen barcode to its corresponding antigen; and
transforming the yeast display vectors into Saccharomyces cerevisiae cells, wherein the S. cerevisiae cells induce surface expression of the yeast display vectors, thereby obtaining the yeast display library expressing the plurality of antigens with the unique antigen barcodes.

In some embodiments, the host receptor protein display library is created by extracting total RNA from human epithelial cell models. For example, ACE2 and DPP4 are amplified from cDNA synthesized using RNA isolated from high glucose-treated Calu-3 cells, a lung epithelial cell line known to express these receptors. RNA is first purified using the RNeasy mini prep kit, followed by cDNA synthesis using Superscript IV reverse transcriptase and oligodT primers. The resulting cDNA serves as a template for PCR amplification of receptor-encoding genes. Primers are designed based on the annotated sequences from the hORFeome v9.1 human genome reference. In parallel, total RNA is also isolated from primary bronchial epithelial cells to amplify an additional set of genes, including other epithelial cell-expressed receptor proteins and internal control genes. These human receptor gene amplicons are then cloned into a custom-designed cell-free display vector equipped with a unique receptor protein barcode sequence. This enables each human receptor protein to be uniquely tracked. Following cloning, the plasmid pool is transformed into electrocompetent E. coli cells to generate a plurality of plasmids.

In some aspects, the eukaryotic cells are yeast cells. In some aspects, the yeast are Saccharomyces cerevisiae cells.

In some embodiments, a barcoded antigen yeast surface display library is constructed for the simultaneous screening of antigen interactions with barcoded host receptor proteins or antibodies. An oligonucleotide pool comprising (i) a degenerate barcode sequence (e.g., WSWSWSWSWSWSWSWSWSAGGAWSWSWSWSWSWSWSWSWS, where W=A/T and S=G/C), (ii) a complement of a 10X template switching oligo (TSO) sequence, and (iii) a complement of the truncated Truseq small RNA read 1 sequence, is amplified using polymerase chain reaction (PCR). This oligo construct is inserted into a yeast surface display vector such as pETcon(-) using Gibson assembly.

In some embodiments, prior to barcode insertion, antigen coding sequences (e.g., viral proteins such as influenza hemagglutinin, SARS-CoV-2 spike RBD, or HIV-1 gp120) are cloned into the yeast display vector upstream of the barcode sequence. The resulting barcoded plasmid library is transformed into yeast cells, such as Saccharomyces cerevisiae EBY100, and induced for surface expression in galactose-containing selective medium. Antigens are typically expressed as C-terminal fusion proteins to the yeast Aga2p anchor protein. An epitope tag, such as a His-tag, facilitates surface expression validation via flow cytometry using a fluorophore-conjugated anti-His antibody.

Also disclosed herein is a method for simultaneous detection of a host receptor protein and a neutralizing antibody that blocks the interaction of said host receptor protein with an antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a receptor protein-barcode dictionary by mapping each unique receptor protein barcode to its corresponding host receptor protein;
expressing the plurality of plasmids in a cell-free system; thereby obtaining barcoded host receptor proteins;
constructing a barcoded antigen library comprising a plurality of plasmids encoding a plurality of antigens;
expressing the plurality of antigens in a cell culture; wherein each antigen is associated with a unique antigen barcode, thereby obtaining barcoded antigens;
contacting B cells to the barcoded antigens and a barcoded host receptor protein-barcoded antigen complex;
sorting B cells bound to the barcoded antigens and/or the barcoded host receptor protein-barcoded antigen complex;
partitioning the sorted B cells into single-cell emulsions;
introducing a unique cell barcode-labeled bead into each single-cell emulsion;
synthesizing cDNA from each sorted B cell;
amplifying immunoglobulin variable region sequences (V(D)J) sequences, from each B cell with the unique antigen barcode and the unique receptor protein barcode, wherein the V(D)J sequences encode the antigen-binding regions of B cell receptors (BCRs) or secreted antibodies;
performing PCR amplification reactions to generate a plurality of amplicons comprising: (1) the unique cell barcode, a unique molecular identifier (UMI), and the unique antigen barcode, and (2) the unique cell barcode, the unique receptor protein barcode, and the unique molecular identifier (UMI);
sequencing the plurality of amplicons and removing sequences lacking any of the unique cell barcode, the UMI, the unique antigen barcode, or the unique receptor protein barcode;
constructing a UMI count matrix comprising the unique cell barcode, the unique receptor protein barcode, and the unique antigen barcode;
calculating a LIBRA-seq score for each combination comprising the unique cell barcode, the unique receptor protein barcode, and the unique antigen barcode based on the UMI count matrix; and
identifying the neutralizing antibody that blocks the interaction of the host receptor protein with an antigen.

Also, disclosed herein is a method for simultaneous detection of a host receptor protein and a neutralizing antibody that blocks the interaction of said host receptor protein with an antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein
generating a receptor protein-barcode dictionary by mapping each unique receptor protein barcode to its corresponding host receptor protein;
expressing the plurality of plasmids in a cell-free system; thereby obtaining barcoded host receptor proteins;
constructing a barcoded antigen library comprising a plurality of plasmids encoding a plurality of antigens;
expressing the plurality of antigens in a cell culture; wherein each antigen is associated with a unique antigen barcode, thereby obtaining barcoded antigens;
contacting B cells to the barcoded antigens and a barcoded host receptor protein-barcoded antigen complex;
sorting B cells bound to the barcoded antigens and/or the barcoded host receptor protein-barcoded antigen complex;
partitioning the sorted B cells into single-cell emulsions;
introducing a unique cell barcode-labeled bead into each single-cell emulsion;
synthesizing cDNA from each sorted B cell;
amplifying immunoglobulin variable region sequences (V(D)J) sequences, from each B cell with the unique antigen barcode and the unique receptor protein barcode, wherein the V(D)J sequences encode the antigen-binding regions of B cell receptors (BCRs) or secreted antibodies;
performing PCR amplification reactions to generate a plurality of amplicons comprising: (1) the unique cell barcode, a unique molecular identifier (UMI), and the unique antigen barcode, and (2) the unique cell barcode, the unique receptor protein barcode, and the unique molecular identifier (UMI); and
sequencing the plurality of amplicons.

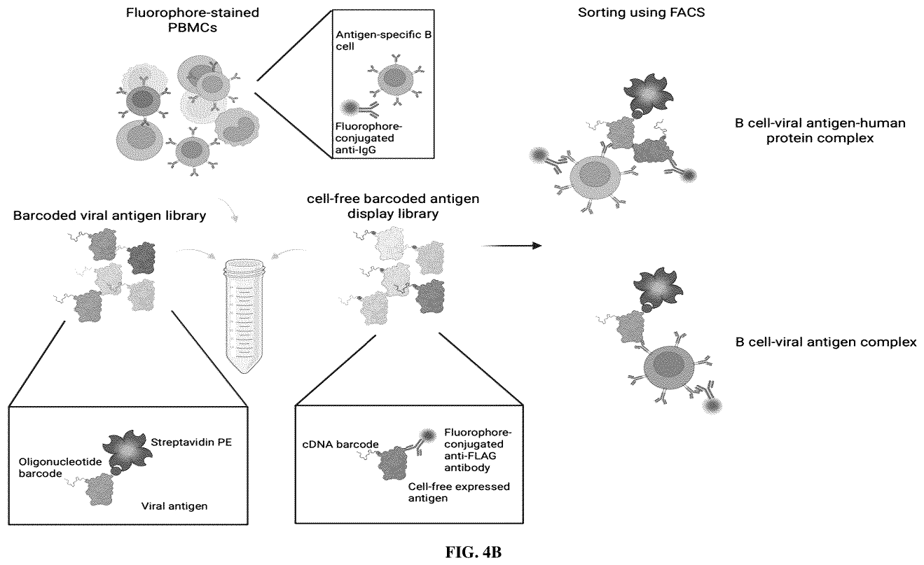

In some aspects, PBMCs are extracted from patients with active infections and/or healthy individuals previously exposed to the pathogen for high-throughput simultaneous identification of host (human) receptors and potently neutralizing antibodies against the pathogen via ligand blocking as defined in FIG. 4B. PBMCs are fluorophore-stained and mixed with the barcoded antigen library and the barcoded host protein library. B cells (CD3⁻, CD14⁻, CD19⁺, IgM⁻, IgG⁺) bound to the antigens as well as to host (human) protein-antigen complexes are sorted. The rationale for sorting the two populations is that when an antibody binds to the antigen alone but not to the complex, the antibody might be binding to an epitope that prevents the interaction between the antigen and the host protein.

In some aspects, the cellular cDNA from B cells are amplified using primers that flank the V(D)J genes in the B cell receptor (BCR). The oligonucleotide with the antigen barcode and cDNA barcode with the host protein is amplified using custom primers. Following purification and sequencing of the antibodies, antigen, and host protein barcodes, the output fastq files are processed using Cell Ranger to assemble, quantify, and annotate the barcode sequences on a cell-by-cell basis.

In some aspects, each antibody-antigen barcode UMI count matrix and host protein barcode UMI count matrix is created and CLR is calculated. This allows the identification of antigen-host receptor pairs, along with antibodies that are targeting epitopes outside of the respective receptor/protein binding site, as well as antibodies that can block the antigens from interacting with the respective host proteins. As defined in FIG. 4B, the variable heavy and light chain sequences of the shortlisted antibodies are cloned into expression vectors with constant heavy and light chain sequences and co-transfected into mammalian Expi293F cells using Expifectamine for micro-expression of IgG. ELISA is performed to confirm the binding of the antibodies to recombinantly expressed antigens. In addition to traditional ELISA, a host protein binding inhibition assay is performed to confirm the host protein and the “functionally relevant” antibody isolated from the platform share the same epitope on the antigen.

In other embodiments, barcoded antigen libraries are generated via recombinant protein expression. Plasmids encoding Avi-tagged antigens are transiently transfected into mammalian cells, such as Expi293F cells, using a transfection reagent (e.g., Gibco™ ExpiFectamine™ 293). The expressed antigens, which include a His epitope tag, are harvested from the supernatant and purified using affinity chromatography followed by size exclusion chromatography. The purified Avi-tagged antigens are enzymatically biotinylated using the BirA enzyme and conjugated to streptavidin-phycoerythrin (PE) carrying a unique DNA barcode. This allows multiplexed antigen detection and quantification during downstream single-cell assays. Examples of antigens that may be used include full-length SARS-CoV-2 spike protein, hepatitis B surface antigen (HBsAg), or HIV-1 gp140. The combination of yeast-displayed and recombinantly expressed barcoded antigen libraries provides versatility for capturing a broad range of antibody-antigen or receptor-antigen interactions in high-throughput discovery.

In some aspects, the barcode-labeled antigens are labeled with a first barcode comprising a DNA sequence or an RNA sequence. In some embodiments, the barcode-labeled antigens are labeled with a first barcode comprising a DNA sequence. In some embodiments, the barcode-labeled antigens are labeled with a first barcode comprising an RNA sequence.

In some aspects, the cell barcode-labeled beads are labeled with a second barcode comprising a DNA sequence or an RNA sequence. In some embodiments, the cell barcode-labeled beads are labeled with a second barcode comprising a DNA sequence. In some embodiments, the cell barcode-labeled beads are labeled with a second barcode comprising an RNA sequence. In some embodiments, the cell barcode-labeled beads are labeled with a barcode on the inside of the bead. In some embodiments, the cell barcode-labeled beads are labeled with a barcode encapsulated within the bead. In some embodiments, the cell barcode-labeled beads are labeled with a barcode on the outside of the bead.

These oligos, which contain the cell barcode, both: (1) enable amplification of cellular mRNA transcripts through the template switch oligo that is part of the oligo containing the cell barcode, and (2) directly anneal to the antigen barcode-containing oligos from the antigen. In some embodiments, the oligos delivered from the beads have the general structure: P5_PCR_handle-Cell_barcode-UMI-Template_switch_oligo.

In some aspects, the barcode-labeled proteins comprise a receptor protein associated with viral infection. In some aspects, the receptor proteins comprise human receptor proteins. As used herein, the term “receptor proteins associated with viral infections” refers to a specific molecular component of the cell, which is capable of recognizing and interacting with a virus, and which, after binding to said virus, is capable of generating a signal that initiates a chain of events leading to a biological response. The receptor proteins do not necessarily include a full-length sequence and can refer to a fragment thereof. Such receptor protein may further be a variant sequence formed by amino acid substitution, deletion, or addition or part of a receptor protein, provided its recognition and/or interaction with the viral pathogen is substantially maintained. In some aspects, the human receptor proteins comprise proteins from human epithelial cells.

In some aspects, the method further includes determining a binding score by counting the number of UMIs for each paired Fab sequence.

The current technology provides a number of improvements and benefits over previous technologies. For example, the antigen protein of interest can be tagged with an arbitrary barcode and sequence as an identifier. In addition, the simple presence or absence of a cell barcode is generally not sufficient to identify antigen specificity. Thus, in some embodiments, in addition to a cell barcode, there is also a unique molecular identifier (UMI) on each bead-delivered oligo. Thus, the specificity can be determined based on the number of UMIs per cell barcode:antigen barcode pair.

### EXAMPLES

The following examples are set forth below to illustrate the systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: High-Throughput Simultaneous Identification of Antigen-Antibody Pairs by Screening Antigen-Specific B Cells Using a Cell-Free Barcoded Antigen Display Library

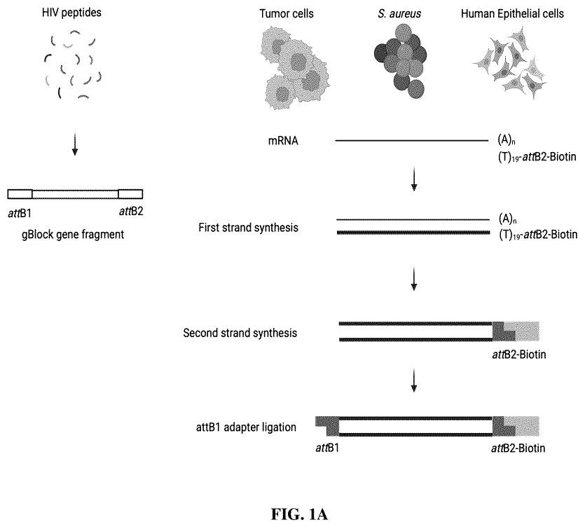

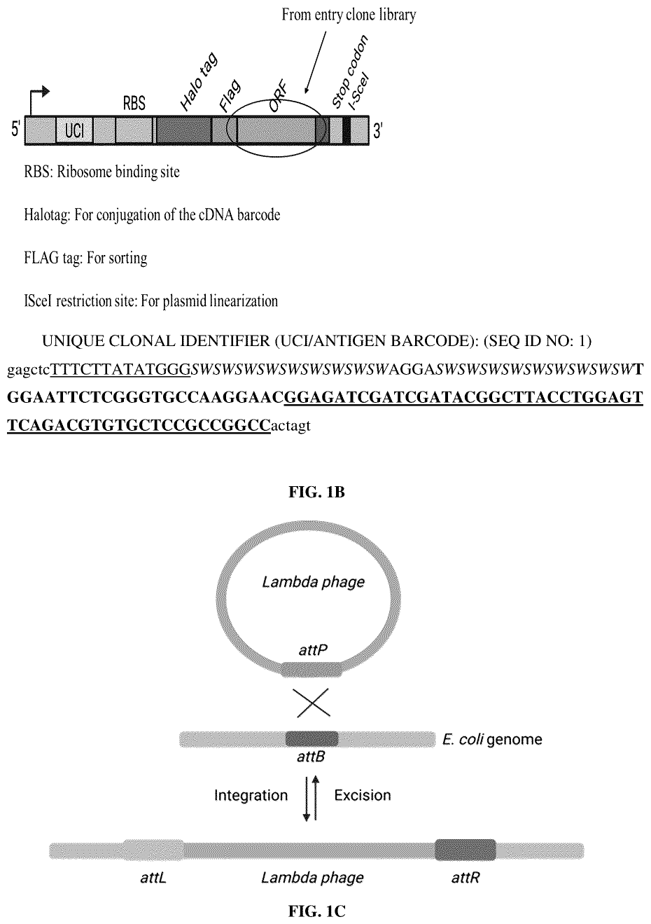

Cell-free barcoded antigen display library generation. The first step in the generation of a cell-free barcoded antigen display library is the generation of a plasmid library encoding peptides or full-length proteins associated with a unique barcode sequence. FIGS. 1A and 1B depict an overview of the formation of cell-free barcoded antigen display libraries. The generation of such a plasmid library enables the expression of tens of thousands of peptides or proteins in a single reaction. The plasmid library is generated using gateway cloning. Gateway cloning involves the construction of two plasmids—entry clone library and destination vector. The open reading frame (ORF) encoding peptides or full-length proteins is cloned into a pDONR™ vector for the construction of the entry clone library. The entry clone library is generated via a BP reaction involving recombination sites, attB1 and attB2 in the ORFs and recombination sites, attP1 and attP2 in the entry vector using Gateway™ BP clonase™ II enzyme (Invitrogen). The entry clone library has the recombination sites attL1 and attL2. The entry clone library construction can be used, for example, in the high-throughput identification of new tumor-associated antigens and neoantigens: total RNA is extracted from early-stage cancer cells/tissues, and the mRNA is reverse transcribed into cDNA and cloned into pDONR™ vector (example, pDONR222 vector) using the CloneMiner II cDNA library construction kit (Invitrogen). In other examples, the entry clone library is used for the identification of antigen-antibody pairs associated with nosocomial bacterial infections: total RNA is extracted from cultures of nosocomial infection-causing bacteria such as C. difficile, S. aureus and A. baumannii using the RNAqueous™ total RNA isolation kit (Invitrogen) and enriched for mRNA from the total RNA using the MICROBExpress™ bacterial mRNA enrichment kit (Invitrogen). Poly(A) tail is then added to the bacterial mRNA using E. coli poly(A) polymerase enzyme. The poly(A) tailed mRNA is reverse transcribed into cDNA and cloned into pDONR™ vector using the CloneMiner II cDNA library construction kit (Invitrogen). In yet another example, the entry clone is used for the identification of antigen- antibody pairs associated with HIV-1: synthesized genes corresponding to HIV-1 envelope peptides that have been shown to elicit a strong immune response are synthesized with attB1 and attB2 sites and cloned into the pDONR™ vector.

The ORFs from the entry clone library are cloned into the destination vector (cell-free display vector with barcodes) via an LR reaction involving recombination sites attL1 and attL2 in the former and recombination sites attR1 and attR2 in the latter using Gateway™ LR clonase™ II enzyme (Invitrogen). Prior to the LR reaction, a custom destination cell-free display vector with barcodes is constructed using a pDEST15 gateway destination vector as the backbone. Gene fragments encoding the T7 promoter, ribosome binding site (RBS), N-terminal HaloTag, FLAG tag, and recombination sequence (attR1 and attR2) for insertion of ORF and IScel restriction site are synthesized and cloned via conventional restriction enzyme digestion and ligation strategy into the pDEST15 vector, replacing the GST tag. An oligonucleotide pool consisting of at least 10 nucleotides. As used herein, is a 40-nucleotide-long degenerate barcode sequence comprising for instance, SWSWSWSWSWSWSWSWSWAGGASWSWSWSWS WSWSWSWSW, where S=G/C; W=A/T), (SEQ ID NO: 1) complement of 10X template switching oligo (TSO) sequence and complement of truncated Truseq small RNA read 1 sequence is amplified using polymerase chain reaction (PCR) and cloned into the destination vector. The degeneracy of the oligonucleotide pool (˜10¹⁰) allows for the addition of unique barcodes to the ORFs. The addition of unique barcodes this way overcomes the need for a separate barcode conjugation step that is normally done for a LIBRA-seq experiment. The use of a separate barcode conjugation step usually requires a huge amount of protein while having a poor recovery rate. Cloning of the ORFs may also be performed by conventional restriction enzyme digestion and ligation or using Gibson/HiFi DNA assembly into the display vector following the addition of the appropriate flanking sequences. Following cloning of the ORFs, the LR reaction or ligation mix is transformed into E. coli electrocompetent cells (such as Electromax DH10B cells) to obtain the cell-free display plasmid library containing ˜10⁶ independent clones.

High throughput identification of antibodies to peptides. From the HIV molecular immunology database, 48 HIV-1 envelope linear epitope sequences are chosen for expression as HIV-1 env peptides using the custom cell-free display system. The linear epitopes are chosen based on their immunogenicity and availability of already known antibodies to the region. Gene fragments to be synthesized as gBlocks (IDT) are designed with flanking attB1 and attB2 sites and a nucleotide sequence of the (G₄S)₃ linker upstream of the peptide sequence. The attB1 and attB2 sites are added for gateway cloning of the genes coding for the peptides into the entry clone vector pDONR221 for subsequent cloning into the custom cell-free barcoded display vector with barcodes (destination vector). An overnight BP clonase reaction is performed to clone the gene fragments into the pDONR221 vector separately. Following clonase reaction, the reaction mix is transformed into One Shot Top10 chemically competent cells and spread on LB agar plates supplemented with Kanamycin. Following overnight incubation, single colonies are picked, and the plasmid is extracted using plasmid mini prep kits. The purified plasmids are then cloned into the cell-free barcoded display vector using an overnight LR clonase reaction. Following LR clonase reaction, the reaction mix is subject to clean-up using PCR and DNA clean-up kit, and the entire mix is transformed into Electromax DH10B competent cells. The transformed bacterial cells are plated onto LB agar plates supplemented with ampicillin and following overnight incubation, a single colony from each plate is taken for plasmid extraction. The clonase reactions are kept separate for each HIV peptide to develop and validate our long-read sequencing approach. The extracted plasmids are sequenced using sangar sequencing as well as long-read sequencing and the results are compared to ensure the long read sequencing approach could give a reliable antigen-barcode dictionary. For long read sequencing, a barcoded cell-free HIV peptide display plasmid library is generated by pooling 45 out of 48 of the barcoded cell-free HIV peptide plasmids and subject to sample preparation for sequencing using Oxford Nanopore's PCR barcoding kit. The sample is then sequenced using a MinION Mk1C sequencer using a minION flow cell and the sequences are base called using guppy super high accuracy model. The pass reads following long read sequencing are analyzed using an analysis pipeline involving nanoQC for sequencing QC, flexiplex for barcode detection, and minimap2 for quick alignment of reads to peptide sequences. Next, a rapid sequencing kit is used for long-read sequencing sample preparation to remove any errors caused due to PCR amplification. DNA barcode-HIV peptide complex is generated and utilized for sorting of antigen-specific B cells from HIV +ve donor PBMCs.

High-throughput identification of antigen-antibody pairs associated with S. aureus infection. Currently, there are no vaccines to prevent nosocomial infections caused by bacteria such as antibiotic-resistant Staphylococcus aureus, Acinetobacter baumannii, and Clostridioides difficile. A successful vaccine to protect against these infections needs to target multiple antigens on the bacteria. dtLIBRA seq focuses on capturing antibody-antigen pairs from PBMCs isolated from patients with active infection and/or patients who have recovered from these infections. The sequence information of the captured antibody-antigen pairs from the approach enables the identification of previously unidentified bacterial proteins involved in eliciting a strong immune response in humans that may then be utilized for the design of better vaccine candidates. The isolated antibodies can be utilized for prophylaxis or as a therapeutic against these bacterial infections.

Isolate total RNA from lab-grown cultures of S. aureus using the RNAqueous total RNA isolation kit and enrich for mRNA from the total RNA using the MICROBExpress bacterial mRNA enrichment kit. Poly(A) tail is then added to the bacterial mRNA using E. coli poly(A) polymerase enzyme. The poly(A) tailed mRNA is reverse transcribed into mRNA and cloned into pDONR222 vector using the CloneMiner II cDNA library construction kit for subsequent gateway cloning into the cell-free display vector. Following cloning into the pDONR222 vector, the BP clonase reaction mix is transformed into Electromax DH10B competent cells and spread on LB agar plates supplemented with Kanamycin. Following overnight incubation, all bacterial colonies are scrapped from the plates and subject to plasmid extraction. The extracted plasmids are pooled and cloned into the custom cell-free display vector with barcodes using an overnight LR clonase reaction for the generation of the barcoded antigen cell-free display plasmid library.

Antigen-barcode dictionary generation. Following the construction of the plasmid library for cell-free display, an antigen-barcode dictionary to identify the barcode associated with each ORF is constructed using the Oxford Nanopore long-read sequencing approach. The long-read sequencing approach can allow for the sequencing of the whole length of the plasmid libraries without the need for assembly. The plasmid library is prepared for long-read sequencing using the Rapid Sequencing kit V14 and loaded onto an R10.4.1 flow cell for sequencing using a Nanopore Mk1C sequencer (or other Nanopore sequencers). Alternatively, the plasmid library is prepared for long read sequencing using a PCR barcoding kit using custom primers and kit-provided primers for amplification of the plasmid libraries and addition of rapid adapters, respectively for sequencing using the Oxford Nanopore MinION Mk1C sequencer (or other Nanopore sequencers). Basecalling of the generated sequences is performed using a high-accuracy model and subject to bioinformatic analysis using Python and R for the construction of the antigen-barcode dictionary. The dictionaries may also be constructed using PacBio or Illumina sequencing when appropriate handles or adapter sequences are added to the oligonucleotide pool consisting of the degenerate barcode sequence.

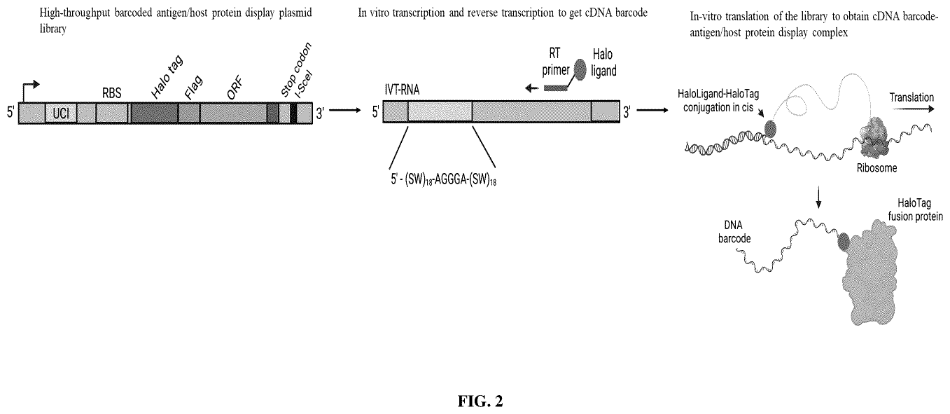

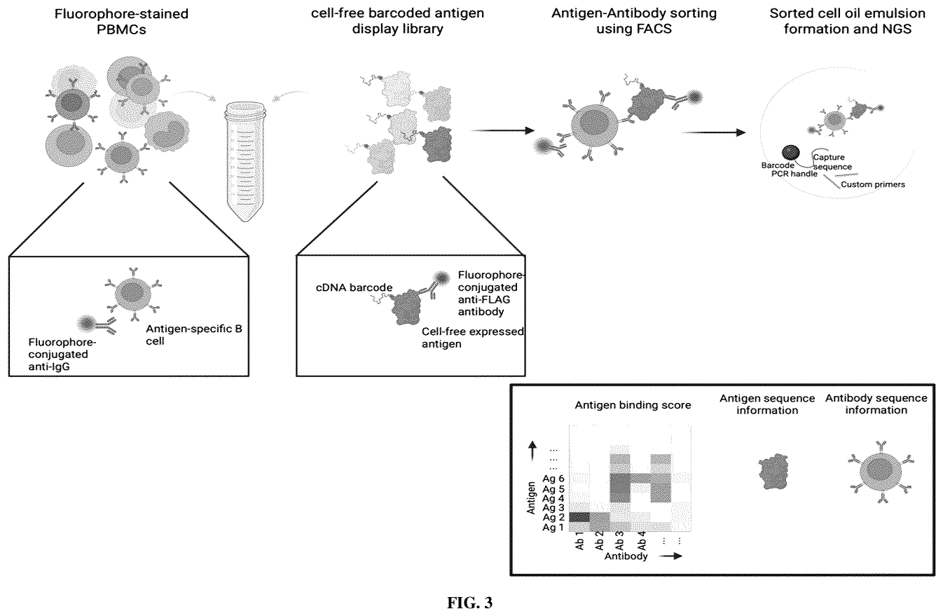

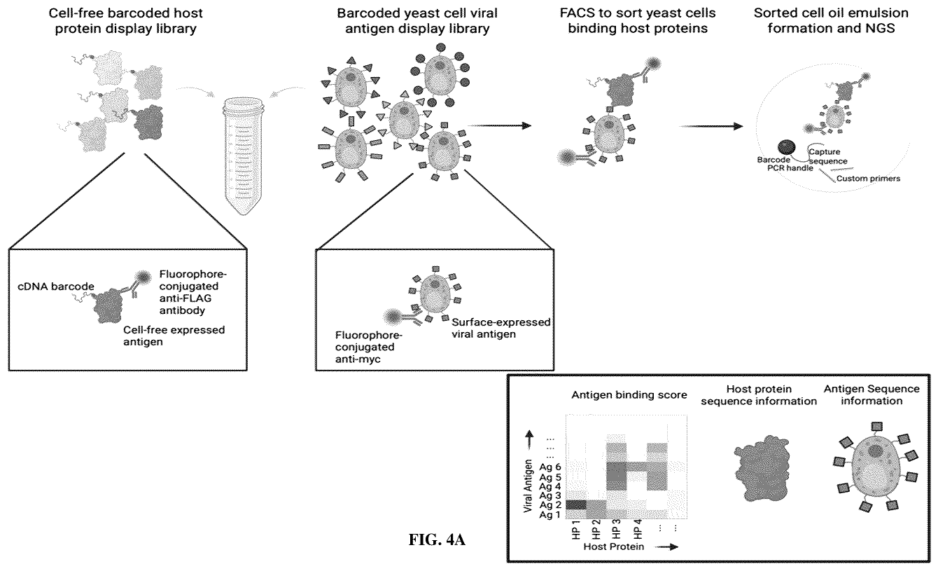

Antigen-specific FACS (FIGS. 2 and 3). Following the construction of the protein-barcode dictionary, the plasmid libraries are transcribed into mRNA using the HiScribe® T7 ARCA mRNA kit (with tailing; New England Biolabs), for instance. The resulting transcribed mRNA consists of a 5′ Cap and a 3′ Poly(A) tail for increased stability and improved translation efficiency. Prior to translation of the transcribed mRNA for expression of the antigens, the barcode sequences are reverse transcribed into cDNA using a custom reverse transcription (RT) primer. The custom RT primer is designed to recognize a site upstream of the RBS and is 5′ amine modified to enable labeling with HaloLigand Succinimidyl Ester (Promega). The HaloLigand facilitates the conjugation of the cDNA barcodes with their associated antigens via the HaloTag following in vitro translation of the cDNA barcoded mRNA transcripts.

Alternatively, other Tags such as (for example, HA, HIS) may be used to capture their ligands (for example, Succinimidyl Ester-modified anti-HA, anti-HIS scFv) for association of the cDNA barcodes with their cell-free expressed antigens. The in vitro translation is performed using the PURExpress A Ribosome Kit, for instance with exogenous ribosomes added to the reaction. Healthy or patient-derived PBMCs are incubated with the cell-free expressed cDNA barcode-antigen complexes in a rotator at 4° C. Following incubation, the complexes are stained with a fluorophore-conjugated antibody (such as a FITC-conjugated anti-FLAG antibody) to the FLAG tag. To sort B cells expressing antigen-specific antibodies from patient/healthy PBMCs that may bind the cell-free expressed cDNA barcode-antigen complex, the PBMCs are stained with a panel of fluorophore-conjugated antibodies specific to cell markers on T cells (CD3), monocytes (CD14) and B cells (CD19, IgG, IgM). The B cells that are live, CD3⁻, CD14⁻, CD19⁺, IgM⁻, and IgG⁺ bound to the cDNA barcode-antigen complex are bulk sorted and resuspended in complete RPMI to 800-1200 cells/μL at viability >90%.

Sequencing and analysis. The sorted cells can then be processed for library preparation using single-cell analysis systems such as 10X Genomics, BD Rhapsody, or others. For library preparation, the sorted cells are partitioned into single-cell suspensions using microfluidic circuits/microwells. The protocol includes a minor modification for the amplification and purification of cDNA barcode and BCR libraries. Briefly, the sorted cells are partitioned into individual cells prior to lysis. Upon lysis, the mRNA from the B cells is reverse transcribed into cDNA and the Next Generation Sequencing (NGS) oligo adaptor present on the beads can capture the V_{L}-V_{H} gene from cDNA of B cells and cDNA barcode on the cDNA barcode-antigen complex and add a unique barcode and unique molecular identifiers (UMI) that can allow for the downstream association of the antigen with the BCR sequence. Alternatively, polyadenylated sequences on mRNAs are captured by the beads, after which cDNA synthesis is performed. The cellular cDNA from B cells and cDNA barcodes on antigens are amplified using custom primers that flank the cDNA antigen barcodes and the V(D)J genes of BCR genes. The resulting amplified libraries are purified using SPRI beads (1.6× purification), and the BCR genes and antigen barcode libraries are sequenced using sequencers such as NovaSeq 6000 with a target of 10000 reads or more per cell. Output fastq files are processed to assemble, quantify, and annotate paired BCR sequences and antigen barcodes on a cell-by-cell basis using barcodes/UMI introduced via the beads. The cDNA barcode sequences associated with each antigen are cross-referenced with the antigen-barcode dictionary to simultaneously discover the antigen that binds a BCR. The amplified libraries may also be sequenced using long-read sequencing approaches following end repair and appropriate adapter ligation.

Binding score determination. To determine the antigen-specificity of a BCR to its associated antigen, each BCR binding a cDNA barcode-antigen complex is given a binding score. The binding score is calculated by counting the number of UMIs for the respective antigen barcode and calculating the centered-log ratio (CLR) of each antigen UMI for each cell. All counts of 1, 2, and 3 UMI are set to 0 to account for background noise.

Validation and/or characterization of new antigens and antibodies. Antigen-specific BCRs associated with a binding score >1 are shortlisted. The variable heavy and light chain sequences of the shortlisted BCR are cloned into expression vectors with constant heavy and light chain sequences and co-transfected into mammalian Expi293F cells using a transfection reagent (example, Expifectamine) for micro-expression of IgG. The antigen sequences can also be cloned into expression vectors (for example, pcDNA3.1(+)) and transfected into mammalian Expi293F cells for expression. The recombinantly expressed antigens are purified using affinity chromatography and size exclusion chromatography. The expressed IgGs in supernatants can then be tested for binding to antigens using ELISA. To perform ELISA, the purified antigens are immobilized at a concentration of 2 μg/ml on Nunc immune 96 well plates. Following immobilization, the wells are blocked and incubated with the supernatants containing antibodies. The wells are washed, and the antigen-antibody binding is detected using HRP (horseradish peroxidase)-conjugated anti-human IgG secondary antibody. Data is collected at an absorbance of 450 nm using a plate reader following the addition of the TMB (3,3′,5,5′-Tetramethylbenzidine) substrate.

### Example 2: High-Throughput Simultaneous Identification of Host (Human) Receptor/Protein Associated With Pathogen Invasion/Infection by Cell-Free Human Receptor/Protein Display Library Using Antigen Yeast Display Library or Recombinantly Expressed Antigens

Cell-free barcoded host (human) protein display library generation. The first step in the generation of a barcoded host protein display library is the generation of a plasmid library encoding proteins associated with a unique barcode sequence. FIGS. 1A and 1B depict an overview of the formation of cell-free barcoded host protein display libraries. The generation of such a plasmid library enables the expression of tens of thousands of proteins in a single reaction. The plasmid library is generated using gateway cloning. Gateway cloning involves the construction of two plasmids—entry clone library and destination vector. The open reading frame (ORF) encoding peptides or full-length proteins is cloned into a pDONR™ vector for the construction of the entry clone library. The entry clone library is generated via a BP reaction involving recombinant sites, attB1 and attB2 in the ORFs and recombination sites, attP1 and attP2 in the entry vector using Gateway™ BP clonase™ II enzyme (Invitrogen). The entry clone library has the recombination sites attL1 and attL2. The entry clone library construction can be used, for example, in the high-throughput identification of host (human) receptors associated with pathogen invasion/infection: cDNA synthesized from total RNA extracted from cultured human mucosal epithelia that form the entry point for most pathogenic organisms, especially viruses is into pDONR™ vector (example, pDONR222 vector) using the CloneMiner II cDNA library construction kit (Invitrogen). The entry clone library can also be the Gateway cloning-adapted CCSB human ORFeome collection. The human ORFeome collection created by the Center for Cancer Systems Biology of the Dana-Farber Institute represents more than 12,000 unique genes (including genes coding for cell surface receptors).

The ORFs from the entry clone library are cloned into the destination vector (custom cell-free display vector with barcodes) via a LR reaction involving recombination sites attL1 and attL2 in the former and recombination sites attR1 and attR2 in the latter using Gateway™ LR clonase™ II enzyme (Invitrogen). Prior to the LR reaction, a custom destination cell-free display vector with barcodes is constructed using a pDEST15 gateway destination vector as the backbone. Gene fragments encoding the T7 promoter, ribosome binding site (RBS), N-terminal HaloTag, FLAG tag, and recombination sequence (attR1 and attR2) for insertion of ORF and IScel restriction site are synthesized and cloned via conventional restriction enzyme digestion and ligation strategy into the pDEST15 vector to replace the GST tag. An oligonucleotide pool consisting of a 40-nucleotide-long degenerate barcode sequence (for instance, SWSWSWSWSWSWSWSWSWAGGASWSWSWSWSWSWSWSWSW, where S=G/C; W=A/T), (SEQ ID NO: 1) complement of 10X template switching oligo (TSO) sequence and complement of truncated Truseq small RNA read 1 sequence is amplified using polymerase chain reaction (PCR) and cloned into the destination vector. The degeneracy of the oligonucleotide pool (˜10¹⁰) allows for the addition of unique barcodes to the ORFs. The addition of unique barcodes this way overcomes the need for a separate barcode conjugation step that is normally done for a LIBRA-seq experiment. The use of a separate barcode conjugation step usually requires a huge amount of protein while having a poor recovery rate. Cloning of the ORFs may also be performed by conventional restriction enzyme digestion and ligation or using Gibson/HiFi DNA assembly into the display vector following the addition of the appropriate flanking sequences. Following cloning of the ORFs, the LR reaction or ligation mix is transformed into E. coli electrocompetent cells (such as Electromax DH10B cells) to obtain the cell-free display plasmid library containing ˜10⁶ independent clones.

High-throughput mapping of viral antigens to their host receptors. To discover a host receptor for a set of diverse coronaviruses that are both known and unknown to infect humans. Even though different coronaviruses have been implicated in interaction with several different host receptors, the receptors for only a small subset of coronaviruses are currently known. This family of viruses is therefore an excellent target for the efforts since it both provides confirmed positive controls (for the known virus-receptor pairs) and an opportunity for new virus-receptor discovery. The successful application of the current technology in this initial test case has paved the way for the development of a general platform for simultaneous virus antigen-host receptor discovery. These efforts offer an unprecedented opportunity to understand the fundamentals of virus-host interactions at an unparalleled scale and across host species and also enable the development of novel therapeutic and preventive modalities, as a first line of defense against both current and emerging viruses.

The first step in the generation of the high-throughput barcoded human host protein display library is the cloning of the gateway-adapted CCSB human ORFeome collection into the custom cell-free display vector. Given the ORFeome collection is expensive, certain genes are amplified from cultured healthy and patient epithelial cells in-house, and attB1 and attB2 sites are added for cloning into the pDONR221 vector. Total RNA from primary healthy bronchial epithelial cells is isolated using RNeasy mini prep kit. cDNA is synthesized from the total RNA using superscript IV reverse transcriptase enzyme with oligo dT primer. The synthesized cDNA is then used as the template for PCR amplification of 13 different genes known to be expressed in healthy epithelial cells. These 13 genes include genes coding for host proteins for non-coronaviruses viruses as well as genes for amplification control. For amplification of the genes, primers are designed based on the annotated sequences of the human genome (hORFeome v9.1). Following PCR amplification of the 13 genes, the amplification reaction is run in a DNA gel, and the gel is purified. ACE2 and DPP4 genes are obtained by amplifying them from cDNA synthesized using total RNA extracted from high glucose-treated Calu-3 cells. The gel-purified amplified genes are gateway-cloned into pDONR221, and their sequence is verified. All 15 genes in the entry vector are cloned into the custom cell-free display vector with barcodes for the generation of the barcoded cell-free host protein display plasmid library. A host protein-barcode dictionary is generated using the Oxford Nanopore long-read sequencing approach.

Simultaneously, a yeast viral protein display library is generated. For the yeast viral protein display library generation, genes corresponding to full-length coronavirus (SARS-CoV-2, MERS and SARS-CoV-2) proteins synthesized as full-length gene fragments (such as gBlocks™ genes fragments) with attB recombination sites are gateway cloned into an entry vector (pDONR221) and later into a custom yeast surface display vector with barcodes, to be expressed as a C-terminus fusion protein to the anchor yeast protein Aga2p. To construct the custom yeast surface display vector with barcodes, an oligonucleotide pool with degenerate barcode sequence (e.g., (WS)₁₈AGGA(WS)₁₈, where W=A/T, S=G/C), (SEQ ID NO: 2) complement of 10X template switching oligo (TSO) sequence and complement of truncated Truseq small RNA read 1 sequence is amplified using polymerase chain reaction (PCR) and assembled into the yeast surface display vector such as pETcon(-) using conventional restriction enzyme digestion and ligation or Gibson assembly. A viral antigen-barcode dictionary is generated. The barcoded yeast surface antigen display plasmid library is transformed into yeast cells such as S. cerevisiae EBY100 and induces surface expression of the antigens in galactose-rich selective media. An epitope tag such as a His tag on the fusion proteins allows the detection of surface expression in FACS using a fluorophore conjugated anti-His antibody.

Following the conversion of the plasmid library into DNA barcode-host protein complexes, the complexes are mixed with recombinantly expressed and barcoded antigens such as SARS-CoV-2, MERS, and SARS-CoV-1 spike proteins and sorted. The sorted cells are processed, sequenced, and analyzed to find host protein-antigen pairs. The ability of the proposed platform to identify the already known binding pairs: SARS-CoV-2 spike-ACE2, SARS-CoV-1 spike-ACE2, and MERS-DPP4 provides the necessary proof-of-concept.

As an alternate approach, the construction of a gateway-adapted cDNA library from mRNA extracted from cultured epithelial cells using a Cloneminer II cDNA library construction kit can also be utilized.

Host protein-barcode dictionary generation. Following the construction of the plasmid library for cell-free display, a host protein-barcode dictionary to identify the barcode associated with each ORF is constructed using the Oxford Nanopore long-read sequencing approach. The long-read sequencing approach can allow for the sequencing of the whole length of the plasmid libraries without the need for assembly. The plasmid library is prepared for long-read sequencing using the Rapid Sequencing kit V14 and loaded onto an R10.4.1 flow cell for sequencing using a Nanopore MinION Mk1C sequencer. Alternatively, the plasmid library is prepared for long-read sequencing using a PCR barcoding kit using custom primers and kit-provided primers for amplification of the plasmid libraries and the addition of rapid adapters, respectively for sequencing using the Oxford Nanopore MinION Mk1C sequencer. Basecalling of the generated sequences is performed using a high-accuracy model and subject to bioinformatic analysis using Python and R for the construction of the host protein-barcode dictionary. The dictionaries may also be constructed using PacBio or Illumina sequencing when appropriate handles or adapter sequences are added to the oligonucleotide pool consisting of the degenerate barcode sequence.

Barcoded Antigen yeast display library generation. For the generation of a barcoded antigen yeast surface display library, an oligonucleotide pool with degenerate barcode sequence (e.g., WSWSWSWSWSWSWSWSWSAGGAWSWSWSWSWSWSWSWSWS, where W=A/T, S=G/C), (SEQ ID NO: 2) complement of 10X template switching oligo (TSO) sequence and complement of truncated Truseq small RNA read 1 sequence is amplified using polymerase chain reaction (PCR) and assembled into the yeast surface display vector such as pETcon(-) using Gibson assembly. Prior to insertion of the barcode, attR1, and attR2 sites for gateway cloning of antigen genes (for example, viral proteins) are inserted into the vector. Following gateway cloning of the antigen genes, the barcodes are cloned into the plasmid library via Gibson/HiFi DNA assembly. An antigen-barcode dictionary like the host protein-barcode dictionary is generated. The barcoded yeast surface antigen display plasmid library transforms into yeast cells such as S. cerevisiae EBY100 and induced for surface expression of the antigens in galactose-rich selective media. The antigens are expressed as C-terminal fusion proteins to the anchor yeast protein Aga2p. An epitope tag such as a His tag on the fusion proteins allows the detection of surface expression in FACS using a fluorophore conjugated anti-His antibody.

Barcoded antigen library can also be prepared by recombinant expression. Plasmids encoding Avi-tagged antigens are transiently transfected into Expi293F cells using Gibco™ ExpiFectamine™ 293 transfection kit for expression. The constructs have a His epitope tag for detection downstream. The expressed antigens are then purified from the cell culture supernatants using affinity chromatography and size exclusion chromatography. The purified Avi-tagged antigens are biotinylated using the BirA enzyme. Streptavidin-PE linked to a unique oligonucleotide/DNA barcode are conjugated to the antigens via biotin.

Antigen-specific FACS (FIGS. 2 and 4A). Following the construction of the protein-barcode dictionary, the plasmid libraries are transcribed into mRNA using the HiScribe® T7 ARCA mRNA kit (with tailing; New England Biolabs), for instance. The resulting transcribed mRNA consists of a 5′ Cap and a 3′ Poly(A) tail for increased stability and improved translation efficiency. Prior to translation of the transcribed mRNA for expression of the antigens, the barcode sequences are reverse transcribed into cDNA using a custom reverse transcription (RT) primer. The custom RT primer is designed to recognize a site upstream of the RBS and is 5′ amine modified to enable labeling with HaloLigand Succinimidyl Ester (Promega). The HaloLigand facilitates the conjugation of the cDNA barcodes with their associated antigens via the HaloTag following in vitro translation of the cDNA barcoded mRNA transcripts. Alternatively, other Tags such as (for example, HA, HIS) may be used to capture their ligands (for example, Succinimidyl Ester-modified anti-HA, anti-HIS scFv) for association of the cDNA barcodes with their cell-free expressed antigens. The in vitro translation is performed using the PURExpress Δ Ribosome Kit, for instance with exogenous ribosomes added to the reaction. The antigen-expressing yeast cells/barcoded recombinantly expressed antigens are then mixed and incubated with the cell-free barcoded host protein display library in a rotator at 4□C. Following incubation, the complexes are stained with a fluorophore-conjugated antibody (such as FITC-conjugated anti-FLAG antibody) to the FLAG tag and a fluorophore-conjugated antibody (such as APC anti-His tag antibody) to the His-tag on the yeast cells (in the case of recombinantly expressed antigens, they are already conjugated with PE). To collect host proteins bound to yeast cells/recombinant antigen, cells that are live and positive for the two fluorophores, one on the anti-FLAG tag antibody and the other on the anti-His tag antibody are sorted, resuspended in DMEM media to 800-1200 cells/μL at a viability of >90%. The sorted cells are subject to subsequent library preparation, sequencing, and analysis.

For high-throughput simultaneous identification of host (human) receptors and potently neutralizing antibodies against the pathogen via ligand blocking (FIG. 4B), fluorophore-stained PBMCs from patients with active infection and/or healthy individuals previously exposed to the pathogen are mixed with the barcoded antigen library and the barcoded host protein library. B cells (CD3⁻, CD14⁻, CD19⁺, IgM⁻, IgG⁺) bound to the antigens as well as to host (human) protein-antigen complexes are sorted. The rationale for sorting the two populations is that when an antibody binds to the antigen alone but not to the complex, the antibody might be binding to an epitope that prevents the interaction between the antigen and the host protein.

Sequencing and analysis. The sorted cells can then be processed for library preparation using single-cell analysis systems such as 10X Genomics, BD Rhapsody, or others. For library preparation, the sorted cells are partitioned into single-cell suspensions using microfluidic circuits/microwells as per the manufacturer's protocol. The protocol includes a minor modification for the amplification and purification of human protein barcode libraries and antigen yeast libraries/recombinantly expressed and barcoded antigens. Briefly, the sorted cells are partitioned into individual cells prior to lysis. Zymolase is added for the lysis of yeast cells. Upon lysis, the mRNA from the yeast cells is reverse transcribed into cDNA and the Next Generation Sequencing (NGS) oligo adaptor present on the beads can capture the yeast cell barcode and the cDNA barcode on the cDNA barcode-host protein complex (and in some cases, cDNA on the recombinantly expressed antigens) and add a unique barcode and unique molecular identifiers (UMI) that can allow for the downstream association of human protein with antigen sequence. Alternatively, polyadenylated sequences on mRNAs are captured by the beads, after which cDNA synthesis is performed. The barcodes on the yeast cells and the cDNA barcodes on the host protein complex are amplified using custom primers. The resulting amplified libraries are purified using SPRI beads (1.6× purification), and antigen and human protein barcode libraries are sequenced using sequencers such as NovaSeq 6000 with a target of at least 10000 reads per cell. Output fastq files are processed to assemble, quantify, and annotate paired antigen sequences and human protein barcodes on a cell-by-cell basis using the barcodes/UMI introduced from the beads. The human protein barcode sequences associated with each human protein are cross-referenced with the human protein-barcode dictionary to simultaneously discover the human protein the antigens can bind. The amplified libraries may also be sequenced using long-read sequencing approaches following end repair and appropriate adapter ligation.

For high-throughput simultaneous identification of host (human) receptors and potently neutralizing antibodies against the pathogen via ligand blocking (FIG. 4B), the cellular cDNA from B cells are amplified using primers that flank the V(D)J genes in the B cell receptor (BCR). The oligonucleotide with the antigen barcode and cDNA barcode with the host protein is amplified using custom primers. Following purification and sequencing of the antibodies, antigen, and host protein barcodes, the output fastq files are processed using Cell Ranger to assemble, quantify, and annotate the barcode sequences on a cell-by-cell basis.

Binding score determination. To determine the specificity of an antigen to its associated host protein, each antigen-binding cDNA barcode-host protein complex is given a binding score. The binding score is calculated by counting the number of UMIs for the respective host protein and calculating the centered-log ratio (CLR) of each host protein UMI for each cell. All counts of 1, 2, and 3 UMI are set to 0 to account for background noise.

With regards to high-throughput simultaneous identification of host (human) receptors and potently neutralizing antibodies against the pathogen via ligand blocking (FIG. 4B), for each antibody, antigen barcode UMI count matrix and host protein barcode UMI count matrix is created and CLR is calculated. This allows the identification of antigen-host receptor pairs, along with antibodies that are targeting epitopes outside of the respective receptor/protein binding site, as well as antibodies that can block the antigens from interacting with the respective host proteins.

Validation and/or characterization of antigens that may bind human proteins. Antigens and human proteins binding with a score >1 are shortlisted. The antigen sequences (as gBlock gene fragments, for instance) are cloned into expression vectors (example, pcDNA3.1(+)) and transfected into mammalian Expi293F cells using a transfection reagent (example, Expifectamine) for recombinant expression of antigens. Similarly, host (human) proteins can also be recombinantly expressed. The recombinantly expressed antigens and human proteins are purified using affinity chromatography and size exclusion chromatography. The expressed antigens can then be tested for binding human proteins using ELISA. To perform ELISA, the purified antigens are immobilized at a concentration of 2 μg/ml on Nunc immune 96 well plates. Following immobilization, the wells are blocked and incubated with the human proteins. The wells are washed, and the antigen-human protein binding is detected using an anti-tag antibody (such as anti-His, based on the tag on the human protein) conjugated to HRP. Data is collected at an absorbance of 450 nm using a plate reader following the addition of the TMB substrate.

With regards to high-throughput simultaneous identification of host (human) receptors and potently neutralizing antibodies against the pathogen via ligand blocking (FIG. 4B), the variable heavy and light chain sequences of the shortlisted antibodies are cloned into expression vectors with constant heavy and light chain sequences and co-transfected into mammalian Expi293F cells using Expifectamine for micro-expression of IgG. ELISA is performed to confirm the binding of the antibodies to recombinantly expressed antigens. In addition to traditional ELISA, a host protein binding inhibition assay is performed to confirm the host protein and the “functionally relevant” antibody isolated from the platform share the same epitope on the antigen.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

SEQ ID NO: 1-UCI for cell-free display vector (Nucleotide sequence)
gagctcTTTCTTATATGGGSWSWSWSWSWSWSWSWSWAGGASWSWSWSWSWSWS
WSWSWTGGAATTCTCGGGTGCCAAGGAACGGAGATCGATCGATACGG
CTTACCTGGAGTTCAGACGTGTGCTCCGCCGGCCactagt
•lowercase-restriction sites
gagctc-SacI
actagt-SpeI
•Underlined-Complement of 10x TSO oligo
•Italics-Barcode; wherein S = G/C and W = A/T
•Bold-complement of truncated TruSeq small RNA read 1 sequence
•Bold and underline-sequence added to increase distance of RT primer from RBS to 32 nt
SEQ ID NO: 2-UCI for PCR and cloning (Nucleotide sequence)
CGACTCACTATAGGGAGACCACgagctcTTTCTTATATGGGSWSWSWSWSWSWS
WSWSWAGGASWSWSWSWSWSWSWSWSWTGGAATTCTCGGGTGCCAAGGA
ACGGAGATCGATCGATACGGCTTACCTGGAGTTCAGACGTGTGCTCC
GCCGGCCactagtAATTTTGTTTAAC
•Italics and underline-Additional sequences for restriction enzyme digestion
•lowercase-restriction sites
gagctc-SacI
actagt-SpeI
•Underlined-Complement of 10x TSO oligo
•Italics-Barcode; wherein S = G/C and W = A/T
•Bold-complement of truncated TruSeq small RNA read 1 sequence
•Bold and underline-sequence added to increase distance of RT primer from RBS to 32 nt
SEQ ID NO: 3-UCI Fwd primer for PCR amplification of the UCI oligos (Nucleotide sequence)
5′ CGACTCACTATAGGGAGACCACGAG 3′
SEQ ID NO: 4-UCI Rev primer for PCR amplification of the UCI oligos(Nucleotide sequence)
5′ GTTAAACAAAATTACTAGTGGCCGGCG 3′
SEQ ID NO: 5-Fwd primer for reverse transcription (Nucleotide sequence)
5′ CTCGGGTGCCAAGGAACGGAGATCGATCGATACGGCTTACCTGGAG 3′
SEQ ID NO: 6-Rev primer for reverse transcription (Nucleotide sequence)
5′ CTCCAGGTAAGCCGTATCGATCGATCTCCGTTCCTTGGCACCCGAG 3′
SEQ ID NO: 7-UCI FOR YEAST SURFACE DISPLAY VECTOR (Nucleotide sequence)
CGACTCACTATAGGGAGACCACactagtTTTCTTATATGGGSWSWSWSWSWSWSW
SWSWAGGASWSWSWSWSWSWSWSWSWTGGAATTCTCGGGTGCCAAGGAA
CGGAGATCGATCGATACGGCTTACCTGGAGTTCAGACGTGTGCTCCG
CCGGCCgctgagcAATTTTGTTTAAC
•Italics and underline-Additional sequences for restriction enzyme digestion
•lowercase-restriction sites
actagt-SpeI
gctgagc-BlpI
•Underlined-Complement of 10x TSO oligo
•Italics-Barcode; wherein S = G/C and W = A/T
•Bold-complement of truncated TruSeq small RNA read 1 sequence
•Bold and underline-sequence added to increase distance of RT primer from RBS to 32 nt
SEQ ID NO: 8-UCI Fwd primer_pETcon (Nucleotide sequence)
5′ CGACTCACTATAGGGAGACCACACTAG 3′
SEQ ID NO: 9-UCI Rev primer_pETcon (Nucleotide sequence)
5′ GTTAAACAAAATTGCTCAGCGGCCGGC 3′
SEQ ID NO: 10-Custom cell-free display vector with UCI/barcode (Nucleotide sequence)
ATCGAGATCTCGATCCCGCGAAATTAATACGACTCACTATAGGGAGACCA
CGAGCTCTTTCTTATATGGGSWSWSWSWSWSWSWSWSWAGGASWSWSW
SWSWSWSWSWSWTGGAATTCTCGGGTGCCAAGGAACGGAGATCGATCGA
TACGGCTTACCTGGAGTTCAGACGTGTGCTCCGCCGGCCACTAGTCCTATA
CAAATAATTTTGTTTAACTTTAAGAAGGAGATATAACCATGGCAGAAATC
GGTACTGGCTTTCCATTCGACCCCCATTATGTGGAAGTCCTGGGCGAGCGC
ATGCACTACGTCGATGTTGGTCCGCGCGATGGCACCCCTGTGCTGTTCCTG
CACGGTAACCCGACCTCCTCCTACGTGTGGCGCAACATCATCCCGCATGTT
GCACCGACCCATCGCTGCATTGCTCCAGACCTGATCGGTATGGGCAAATC
CGACAAACCAGACCTGGGTTATTTCTTCGACGACCACGTCCGCTTCATGGA
TGCCTTCATCGAAGCCCTGGGTCTGGAAGAGGTCGTCCTGGTCATTCACGA
CTGGGGCTCCGCTCTGGGTTTCCACTGGGCCAAGCGCAATCCAGAGCGCG
TCAAAGGTATTGCATTTATGGAGTTCATCCGCCCTATCCCGACCTGGGACG
AATGGCCAGAATTTGCCCGCGAGACCTTCCAGGCCTTCCGCACCACCGAC
GTCGGCCGCAAGCTTATTATCGATCAGAACGTTTTTATCGAGGGTACGCTG
CCGATGGGTGTCGTCCGCCCGCTGACTGAAGTCGAGATGGACCATTACCG
CGAGCCGTTCCTGAATCCTGTTGACCGCGAGCCACTGTGGCGCTTCCCAAA
CGAGCTGCCAATCGCCGGTGAGCCAGCGAACATCGTCGCGCTGGTCGAAG
AATACATGGACTGGCTGCACCAGTCCCCTGTCCCGAAGCTGCTGTTCTGGG
GCACCCCAGGCGTTCTGATCCCACCGGCCGAAGCCGCTCGCCTGGCCAAA
AGCCTGCCTAACTGCAAGGCTGTGGACATCGGCCCGGGTCTGAATCTGCT
GCAAGAAGACAACCCGGACCTGATCGGCAGCGAGATCGCGCGCTGGCTAT
CTACGCTAGAAATTTCCGGCGACTACAAAGACGATGACGACAAGTCGGAT
CTGGTTCCGCGTCCATGGTCGAATCAAACAAGTTTGTACAAAAAAGCTGA
ACGAGAAACGTAAAATGATATAAATATCAATATATTAAATTAGATTTTGC
ATAAAAAACAGACTACATAATACTGTAAAACACAACATATCCAGTCACTA
TGGCGGCCGCATTAGGCACCCCAGGCTTTACACTTTATGCTTCCGGCTCGT
ATAATGTGTGGATTTTGAGTTAGGATCCGTCGAGATTTTCAGGAGCTAAG
GAAGCTAAAATGGAGAAAAAAATCACTGGATATACCACCGTTGATATATC
CCAATGGCATCGTAAAGAACATTTTGAGGCATTTCAGTCAGTTGCTCAATG
TACCTATAACCAGACCGTTCAGCTGGATATTACGGCCTTTTTAAAGACCGT
AAAGAAAAATAAGCACAAGTTTTATCCGGCCTTTATTCACATTCTTGCCCG
CCTGATGAATGCTCATCCGGAATTCCGTATGGCAATGAAAGACGGTGAGC
TGGTGATATGGGATAGTGTTCACCCTTGTTACACCGTTTTCCATGAGCAAA
CTGAAACGTTTTCATCGCTCTGGAGTGAATACCACGACGATTTCCGGCAGT
TTCTACACATATATTCGCAAGATGTGGCGTGTTACGGTGAAAACCTGGCCT
ATTTCCCTAAAGGGTTTATTGAGAATATGTTTTTCGTCTCAGCCAATCCCT
GGGTGAGTTTCACCAGTTTTGATTTAAACGTGGCCAATATGGACAACTTCT
TCGCCCCCGTTTTCACCATGGGCAAATATTATACGCAAGGCGACAAGGTG
CTGATGCCGCTGGCGATTCAGGTTCATCATGCCGTTTGTGATGGCTTCCAT
GTCGGCAGAATGCTTAATGAATTACAACAGTACTGCGATGAGTGGCAGGC
GGGGCGTAATCTAGAGGATCCGGCTTACTAAAAGCCAGATAACAGTATGC
GTATTTGCGCGCTGATTTTTGCGGTATAAGAATATATACTGATATGTATAC
CCGAAGTATGTCAAAAAGAGGTATGCTATGAAGCAGCGTATTACAGTGAC
AGTTGACAGCGACAGCTATCAGTTGCTCAAGGCATATATGATGTCAATAT
CTCCGGTCTGGTAAGCACAACCATGCAGAATGAAGCCCGTCGTCTGCGTG
CCGAACGCTGGAAAGCGGAAAATCAGGAAGGGATGGCTGAGGTCGCCCG
GTTTATTGAAATGAACGGCTCTTTTGCTGACGAGAACAGGGGCTGGTGAA
ATGCAGTTTAAGGTTTACACCTATAAAAGAGAGAGCCGTTATCGTCTGTTT
GTGGATGTACAGAGTGATATTATTGACACGCCCGGGCGACGGATGGTGAT
CCCCCTGGCCAGTGCACGTCTGCTGTCAGATAAAGTCCCCCGTGAACTTTA
CCCGGTGGTGCATATCGGGGATGAAAGCTGGCGCATGATGACCACCGATA
TGGCCAGTGTGCCGGTCTCCGTTATCGGGGAAGAAGTGGCTGATCTCAGC
CACCGCGAAAATGACATCAAAAACGCCATTAACCTGATGTTCTGGGGAAT
ATAAATGTCAGGCTCCCTTATACACAGCCAGTCTGCAGGTCGACCATAGT
GACTGGATATGTTGTGTTTTACAGTATTATGTAGTCTGTTTTTTATGCAAA
ATCTAATTTAATATATTGATATTTATATCATTTTACGTTTCTCGTTCAGCTT
TCTTGTACAAAGTGGTTTGATTCGACCCGGGATCCGGCTGCTAACAAAGC
CCGAAAGGAAGCTGAGTTGGCTGCTGCCACCGCTGAGCAATAACTAGCAT
AACCCCTTGGGGCCTCTAAACGGGTCTTGAGGGGTTTTTTGCTGAAAGGA
GGAACTATATCCGGATATCCACAGGACGGGTGTGGTCGCCATGATCGCGT
AGTCGATAGTGGCTCCAAGTAGCGAAGCGAGCAGGACTGGGCGGCGGCC
AAAGCGGTCGGACAGTGCTCCGAGAACGGGTGCGCATAGAAATTGCATCA
ACGCATATAGCGCTAGCTAGGGATAACAGGGTAATAGCACGCCATAGTGA
CTGGCGATGCTGTCGGAATGGACGATATCCCGCAAGAGGCCCGGCAGTAC
CGGCATAACCAAGCCTATGCCTACAGCATCCAGGGTGACGGTGCCGAGGA
TGACGATGAGCGCATTGTTAGATTTCATACACGGTGCCTGACTGCGTTAGC
AATTTAACTGTGATAAACTACCGCATTAAAGCTTATCGATGATAAGCTGTC
AAACATGAGAATTCTTGAAGACGAAAGGGCCTCGTGATACGCCTATTTTT
ATAGGTTAATGTCATGATAATAATGGTTTCTTAGACGTCAGGTGGCACTTT
TCGGGGAAATGTGCGCGGAACCCCTATTTGTTTATTTTTCTAAATACATTC
AAATATGTATCCGCTCATGAGACAATAACCCTGATAAATGCTTCAATAAT
ATTGAAAAAGGAAGAGTATGAGTATTCAACATTTCCGTGTCGCCCTTATTC
CCTTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTA
TCCCGTGTTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCCTTGATCGTTG
GGAACCGGAGCTGAATGAAGCCATACCAAACGACGAGCGTGACACCACG
ATGCCTGCAGCAATGGCAACAACGTTGCGCAAACTATTAACTGGCGAACT
ACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCA
CTGGGGCCAGATGGTAAGCCCTCCCGTATCGTAGTTATCTACACGACGGG
GAGTCAGGCAACTATGGATGAACGAAATAGACAGATCGCTGAGATAGGT
GCCTCACTGATTAAGCATTGGTAACTGTCAGACCAAGTTTACTCATATATA
CTTTAGATTGATTTAAAACTTCATTTTTAATTTAAAAGGATCTAGGTGAAG
ATCCTTTTTGATAATCTCATGACCAAAATCCCTTAACGTGAGTTTTCGTTCC
ACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCC
GTAGTTAGGCCACCACTTCAAGAACTCTGTAGCACCGCCTACATACCTCGC
TCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGG
GCTGAACGGGGGGTTCGTGCACACAGCCCAGCTTGGAGCGAACGACCTAC
ACCGAACTGAGATACCTACAGCGTGAGCTATGAGAAAGCGCCACGCTTCC
CGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTC
GTCAGGGGGGCGGAGCCTATGGAAAAACGCCAGCAACGCGGCCTTTTTAC
GGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGC
TCGCCGCAGCCGAACGACCGAGCGCAGCGAGTCAGTGAGCGAGGAAGCG
GAAGAGCGCCTGATGCGGTATTTTCTCCTTACGCATCTGTGCGGTATTTCA
CACCGCATATATGGTGCACTCTCAGTACAATCTGCTCTGATGCCGCATAGT
TAAGCCAGTATACACTCCGCTATCGCTACGTGACTGGGTCATGGCTGCGCC
CCGACACCCGCCAACACCCGCTGACGCGCCCTGACGGGCTTGTCTGCTCC
CGGCATCCGCTTACAGACAAGCTGTGACCGTCTCCGGGAGCTGCATGTGT
CAGAGGTTTTCACCGTCATCACCGAAACGCGCGAGGCAGCTGCGGTAAAG
CTCATCAGCGTGGTCGTGAAGCGATTCACAGATGTCTGCCTGTTCATCCGC
GTCCAGCTCGTTGAGTTTCTCCAGAAGCGTTAATGTCTGGCTTCTGATAAA
GCGGGCCATGTTAAGGGCGGTTTTTTCCTGTTTGGTCACTGATGCCTCCGT
GTAAGGGGGATTTCTGTTCATGGGGGTAATGATACCGATGAAACGAGAGA
GGATGCTCACGATACGGGTTACTGATGATGAACATGCCCGGTTACTGGAA
CGTTGTGAGGGTAAACAACTGGCGGTATGGATGCGGCGGGACCAGAGAA
AAATCACTCAGGGTCAATGCCAGCGCTTCGTTAATACAGATGTAGGTGTT
CCACAGGGTAGCCAGCAGCATCCTGCGATGCAGATCCGGAACATAATGGT
GCAGGGCGCTGACTTCCGCGTTTCCAGACTTTACGAAACACGGAAACCGA
AGACCATTCATGTTGTTGCTCAGGTCGCAGACGTTTTGCAGCAGCAGTCGC
TTCACGTTCGCTCGCGTATCGGTGATTCATTCTGCTAACCAGTAAGGCAAC
CCCGCCAGCCTAGCCGGGTCCTCAACGACAGGAGCACGATCATGCGCACC
CGTGGCCAGGACCCAACGCTGCCCGAGATGCGCCGCGTGCGGCTGCTGGA
GATGGCGGACGCGATGGATATGTTCTGCCAAGGGTTGGTTTGCGCATTCA
CAGTTCTCCGCAAGAATTGATTGGCTCCAATTCTTGGAGTGGTGAATCCGT
TAGCGAGGTGCCGCCGGCTTCCATTCAGGTCGAGGTGGCCCGGCTCCATG
CACCGCGACGCAACGCGGGGAGGCAGACAAGGTATAGGGCGGCGCCTAC
AATCCATGCCAACCCGTTCCATGTGCTCGCCGAGGCGGCATAAATCGCCG
TGACGATCAGCGGTCCAGTGATCGAAGTTAGGCTGGTAAGAGCCGCGAGC
GATCCTTGAAGCTGTCCCTGATGGTCGTCATCTACCTGCCTGGACAGCATG
GCCTGCAACGCGGGCATCCCGATGCCGCCGGAAGCGAGAAGAATCATAAT
GGGGAAGGCCATCCAGCCTCGCGTCGCGAACGCCAGCAAGACGTAGCCC
AGCGCGTCGGCCGCCATGCCGGCGATAATGGCCTGCTTCTCGCCGAAACG
TTTGGTGGCGGGACCAGTGACGAAGGCTTGAGCGAGGGCGTGCAAGATTC
CGAATACCGCAAGCGACAGGCCGATCATCGTCGCGCTCCAGCGAAAGCGG
TCCTCGCCGAAAATGACCCAGAGCGCTGCCGGCACCTGTCCTACGAGTTG
CATGATAAAGAAGACAGTCATAAGTGCGGCGACGATAGTCATGCCCCGCG
CCCACCGGAAGGAGCTGACTGGGTTGAAGGCTCTCAAGGGCATCGGTCGA
TCGACGCTCTCCCTTATGCGACTCCTGCATTAGGAAGCAGCCCAGTAGTAG
GTTGAGGCCGTTGAGCACCGCCGCCGCAAGGAATGGTGCATGCAAGGAG
ATGGCGCCCAACAGTCCCCCGGCCACGGGGCCTGCCACCATACCCACGCC
GAAACAAGCGCTCATGAGCCCGAAGTGGCGAGCCCGATCTTCCCCATCGG
TGATGTCGGCGATATAGGCGCCAGCAACCGCACCTGTGGCGCCGGTGATG
CCGGCCACGATGCGTCCGGCGTAGAGG
SEQ ID NO: 11-Custom yeast display library vector with UCI/barcode (Nucleotide sequence)
ATTGTGAGCGGATAACAATTTCACACAGGAAACAGCTATGACCATGATTA
CGCCAAGCTCGAAATTAACCCTCACTAAAGGGAACAAAAGCTGGTACCAA
TTCCTTGAATTTTCAAAAATTCTTACTTTTTTTTTGGATGGACGCAAAGAA
GTTTAATAATCATATTACATGGCATTACCACCATATACATATCCATATCTA
ATCTTACTTATATGTTGTGGAAATGTAAAGAGCCCCATTATCTTAGCCTAA
AAAAACCTTCTCTTTGGAACTTTCAGTAATACGCTTAACTGCTCATTGCTA
TATTGAAGTACGGATTAGAAGCCGCCGAGCGGGTGACAGCCCTCCGAAGG
AAGACTCTCCTCCGTGCGTCCTCGTCTTCACCGGTCGCGTTCCTGAAACGC
AGATGTGCCTCGCGCCGCACTGCTCCGAACAATAAAGATTCTACAATACT
AGCTTTTATGGTTATGAAGAGGAAAAATTGGCAGTAACCTGGCCCCACAA
ACCTTCAAATGAACGAATCAAATTAACAACCATAGGATGATAATGCGATT
AGTTTTTTAGCCTTATTTCTGGGGTAATTAATCAGCGAAGCGATGATTTTT
GATCTATTAACAGATATATAAATGCAAAAACTGCATAACCACTTTAACTA
ATACTTTCAACATTTTCGGTTTGTATTACTTCTTATTCAAATGTAATAAAAG
TATCAACAAAAAATTGTTAATATACCTCTATACTTTAACGTCAAGGAGAA
AAAACCCCGGATCGAATTCGAGACCACACTAGTTTTCTTATATGGGWSWS
WSWSWSWSWSWSWSAGGAWSWSWSWSWSWSWSWSWSTGGAATTCTCG
GGTGCCAAGGAACGGAGATCGATCGATACGGCTTACCTGGAGTTCAGACG
TGTGCTCCGCCGGCCGCTGAGCCCTACTTCATACATTTTCAATTAAGATGC
AGTTACTTCGCTGTTTTTCAATATTTTCTGTTATTGCTTCAGTTTTAGCACA
GGAACTGACAACTATATGCGAGCAAATCCCCTCACCAACTTTAGAATCGA
CGCCGTACTCTTTGTCAACGACTACTATTTTGGCCAACGGGAAGGCAATGC
AAGGAGTTTTTGAATATTACAAATCAGTAACGTTTGTCAGTAATTGCGGTT
CTCACCCCTCAACAACTAGCAAAGGCAGCCCCATAAACACACAGTATGTT
TTTAAGGACAATAGCTCGACGATTGAAGGTAGATACCCATACGACGTTCC
AGACTACGCTCTGCAGACAAGTTTGTACAAAAAAGCTGAACGAGAAACGT
AAAATGATATAAATATCAATATATTAAATTAGATTTTGCATAAAAAACAG
ACTACATAATACTGTAAAACACAACATATCCAGTCACTATGGCGGCCGCA
TTAGGCACCCCAGGCTTTACACTTTATGCTTCCGGCTCGTATAATGTGTGG
ATTTTGAGTTAGGATCCGTCGAGATTTTCAGGAGCTAAGGAAGCTAAAAT
GGAGAAAAAAATCACTGGATATACCACCGTTGATATATCCCAATGGCATC
GTAAAGAACATTTTGAGGCATTTCAGTCAGTTGCTCAATGTACCTATAACC
AGACCGTTCAGCTGGATATTACGGCCTTTTTAAAGACCGTAAAGAAAAAT
AAGCACAAGTTTTATCCGGCCTTTATTCACATTCTTGCCCGCCTGATGAAT
GCTCATCCGGAATTCCGTATGGCAATGAAAGACGGTGAGCTGGTGATATG
GGATAGTGTTCACCCTTGTTACACCGTTTTCCATGAGCAAACTGAAACGTT
TTCATCGCTCTGGAGTGAATACCACGACGATTTCCGGCAGTTTCTACACAT
ATATTCGCAAGATGTGGCGTGTTACGGTGAAAACCTGGCCTATTTCCCTAA
AGGGTTTATTGAGAATATGTTTTTCGTCTCAGCCAATCCCTGGGTGAGTTT
CACCAGTTTTGATTTAAACGTGGCCAATATGGACAACTTCTTCGCCCCCGT
TTTCACCATGGGCAAATATTATACGCAAGGCGACAAGGTGCTGATGCCGC
TGGCGATTCAGGTTCATCATGCCGTTTGTGATGGCTTCCATGTCGGCAGAA
TGCTTAATGAATTACAACAGTACTGCGATGAGTGGCAGGCGGGGCGTAAT
CTAGAGGATCCGGCTTACTAAAAGCCAGATAACAGTATGCGTATTTGCGC
GCTGATTTTTGCGGTATAAGAATATATACTGATATGTATACCCGAAGTATG
TCAAAAAGAGGTATGCTATGAAGCAGCGTATTACAGTGACAGTTGACAGC
GACAGCTATCAGTTGCTCAAGGCATATATGATGTCAATATCTCCGGTCTGG
TAAGCACAACCATGCAGAATGAAGCCCGTCGTCTGCGTGCCGAACGCTGG
AAAGCGGAAAATCAGGAAGGGATGGCATGCGTCGCCCGGTTTATTGAAAT
GAACGGCTCTTTTGCTGACGAGAACAGGGGCTGGTGAAATGCAGTTTAAG
GTTTACACCTATAAAAGAGAGAGCCGTTATCGTCTGTTTGTGGATGTACAG
AGTGATATTATTGACACGCCCGGGCGACGGATGGTGATCCCCCTGGCCAG
TGCACGTCTGCTGTCAGATAAAGTCCCCCGTGAACTTTACCCGGTGGTGCA
TATCGGGGATGAAAGCTGGCGCATGATGACCACCGATATGGCCAGTGTGC
CGGTCTCCGTTATCGGGGAAGAAGTGGCTGATCTCAGCCACCGCGAAAAT
GACATCAAAAACGCCATTAACCTGATGTTCTGGGGAATATAAATGTCAGG
CTCCCTTATACACAGCCAGTCAGCCGGTCGACCATAGTGACTGGATATGTT
GTGTTTTACAGTATTATGTAGTCTGTTTTTTATGCAAAATCTAATTTAATAT
ATTGATATTTATATCATTTTACGTTTCTCGTTCAGCTTTCTTGTACAAAGTG
GTGGATCCGAACAAAAGCTTATTTCTGAAGAGGACTTGTAATAGAGATCT
GATAACAACAGTGTAGATGTAACAAAATCGACTTTGTTCCCACTGTACTTT
TAGCTCGTACAAAATACAATATACTTTTCATTTCTCCGTAAACAACATGTT
TTCCCATGTAATATCCTTTTCTATTTTTCGTTCCGTTACCAACTTTACACAT
ACTTTATATAGCTATTCACTTCTATACACTAAAAAACTAAGACAATTTTAA
TTTTGCTGCCTGCCATATTTCAATTTGTTATAAATTCCTATAATTTATCCTA
TTAGTAGCTAAAAAAAGATGAATGTGAATCGAATCCTAAGAGAATTGAGC
TCCAATTCGCCCTATAGTGAGTCGTATTACAATTCACTGGCCGTCGTTTTA
CAACGTCGTGACTGGGAAAACCCTGGCGTTACCCAACTTAATCGCCTTGC
AGCACATCCCCCTTTCGCCAGCTGGCGTAATAGCGAAGAGGCCCGCACCG
ATCGCCTTTCCCAACAGTTGCGCAGCCTGAATGGCGAATGGACGCGCCCT
GTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGGCTCC
CTTTAGGGTTCCGATTTAGTGCTTTACGGCACCTCGACCCCAAAAAACTTG
ATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAA
CTGGAACAACACTCAACCCTATCTCGGTCTATTCTTTTGATTTATAAGGGA
TTTTGCCGATTTCGGCCTATTGGTTAAAAAATGAGCTGATTTAACAAAAAT
TTAACGCGAATTTTAACAAAATATTAACGCTTACAATTTCCTGATGCGGTA
TTTTCTCCTTACGCATCTGTGCGGTATTTCACACCGCATAGATCGGCAAGT
GCACAAACAATACTTAAATAAATACTACTCAGTAATAACCTATTTCTTAGC
ATTTTTGACGAAATTTGCTATTTTGTTAGAGTCTTTTACACCATTTGTCTCC
ACACCTCCGCTTACATCAACACCAATAACGCCATTTAATCTAAGCGCATCA
CCAACATTTTCTGGCGTCAGTCCACCAGCTAACATAAAATGTAAGCTTTCG
GGGCTCTCTTGCCTTCCAACCCAGTCAGAAATCGAGTTCCAATCCAAAAGT
TCACCTGTCCCACCTGCTTCTGAATCAAACAAGGGAATAAACGAATGAGG
TTTCTGTGAAGCTGCACTGAGTAGTATGTTGCAGTCTTTTGGAAATACGAG
TCTTTTAATAACTGGCAAACCGAGGAACTCTTGGTATTCTTGCCACGACTC
ATCTCCATGCAGTTGGACGATATCAATGCCGTAATCATTGACCAGAGCCA
AAACATCCTCCTTAGGTTGATTACGAAACACGCCAACCAAGTATTTCGGA
GTGCCTGAACTATTTTTATATGCTTTTACAAGACTTGAAATTTTCCTTGCAA
TAACCGGGTCAATTGTTCTCTTTCTATTGGGCACACATATAATACCCAGCA
AGTCAGCATCGGAATCTAGAGCACATTCTGCGGCCTCTGTGCTCTGCAAG
CCGCAAACTTTCACCAATGGACCAGAACTACCTGTGAAATTAATAACAGA
CATACTCCAAGCTGCCTTTGTGTGCTTAATCACGTATACTCACGTGCTCAA
TAGTCACCAATGCCCTCCCTCTTGGCCCTCTCCTTTTCTTTTTTCGACCGAA
TTAATTCTTAATCGGCAAAAAAAGAAAAGCTCCGGATCAAGATTGTACGT
AAGGTGACAAGCTATTTTTCAATAAAGAATATCTTCCACTACTGCCATCTG
GCGTCATAACTGCAAAGTACACATATATTACGATGCTGTTCTATTAAATGC
TTCCTATATTATATATATAGTAATGTCGTGATCTATGGTGCACTCTCAGTA
CAATCTGCTCTGATGCCGCATAGTTAAGCCAGCCCCGACACCCGCCAACA
CCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCA
TCACCGAAACGCGCGAGACGAAAGGGCCTCGTGATACGCCTATTTTTATA
GGTTAATGTCATGATAATAATGGTTTCTTAGACGGATCGCTTGCCTGTAAC
TTACACGCGCCTCGTATCTTTTAATGATGGAATAATTTGGGAATTTACTCT
GTGTTTATTTATTTTTATGTTTTGTATTTGGATTTTAGAAAGTAAATAAAGA
AGGTAGAAGAGTTACGGAATGAAGAAAAAAAAATAAACAAAGGTTTAAA
AAATTTCAACAAAAAGCGTACTTTACATATATATTTATTAGACAAGAAAA
GCAGATTAAATAGATATACATTCGATTAACGATAAGTAAAATGTAAAATC
ACAGGATTTTCGTGTGTGGTCTTCTACACAGACAAGATGAAACAATTCGG
CATTAATACCTGAGAGCAGGAAGAGCAAGATAAAAGGTAGTATTTGTTGG
CGATCCCCCTAGAGTCTTTTACATCTTCGGAAAACAAAAACTATTTTTTCT
TTAATTTCTTTTTTTACTTTCTATTTTTAATTTATATATTTATATTAAAAAAT
TTAAATTATAATTATTTTTATAGCACGTGATGAAAAGGACCCAGGTGGCA
CTTTTCGGGGAAATGTGCGCGGAACCCCTATTTGTTTATTTTTCTAAATAC
ATTCAAATATGTATCCGCTCATGAGACAATAACCCTGATAAATGCTTCAAT
AATATTGAAAAAGGAAGAGTATGAGTATTCAACATTTCCGTGTCGCCCTT
ATTCCCTTTTTTGCGGCATTTTGCCTTCCTGTTTTTGCTCACCCAGAAACGC
TGGTGAAAGTAAAAGATGCTGAAGATCAGTTGGGTGCACGAGTGGGTTAC
ATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGT
ATTATCCCGTATTGACGCCGGGCAAGAGCAACTCGGTCGCCGCATACACT
ATTCTCAGAATGACTTGGTTGAGTACTCACCAGTCACAGAAAAGCATCTT
ACGGATGGCATGACAGTAAGAGAATTATGCAGTGCTGCCATAACCATGAG
TGATAACACTGCGGCCAACTTACTTCTGACAACGATCGGAGGACCGAAGG
AGCTAACCGCTTTTTTGCACAACATGGGGGATCATGTAACTCGCCTTGATC
GTTGGGAACCGGAGCTGAATGAAGCCATACCAAACGACGAGCGTGACAC
CACGATGCCTGTAGCAATGGCAACAACGTTGCGCAAACTATTAACTGGCG
AACTACTTACTCTAGCTTCCCGGCAACAATTAATAGACTGGATGGAGGCG
GATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGC
AGCACTGGGGCCAGATGGTAAGCCCTCCCGTATCGTAGTTATCTACACGA
CGGGGAGTCAGGCAACTATGGATGAACGAAATAGACAGATCGCTGAGAT
AGGTGCCTCACTGATTAAGCATTGGTAACTGTCAGACCAAGTTTACTCATA
TATACTTTAGATTGATTTAAAACTTCATTTTTAATTTAAAAGGATCTAGGT
GAAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCGA
AGGTAACTGGCTTCAGCAGAGCGCAGATACCAAATACTGTTCTTCTAGTG
TAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGC
GGTCGGGCTGAACGGGGGGTTCGTGCACACAGCCCAGCTTGGAGCGAACG
ACCTACACCGAACTGAGATACCTACAGCGTGAGCTATGAGAAAGCGCCAC
GCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGTG
ATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCTG
CGTTATCCCCTGATTCTGTGGATAACCGTATTACCGCCTTTGAGTGAGCTG
ATACCGCTCGCCGCAGCCGAACGACCGAGCGCAGCGAGTCAGTGAGCGA
GGAAGCGGAAGAGCGCCCAATACGCAAACCGCCTCTCCCCGCGCGTTGGC
CGATTCATTAATGCAGCTGGCACGACAGGTTTCCCGACTGGAAAGCGGGC
AGTGAGCGCAACGCAATTAATGTGAGTTAGCTCACTCATTAGGCACCCCA
GGCTTTACACTTTATGCTTCCGGCTCGTATGTTGTGTGGA

What is claimed is:

1. A method for simultaneous detection of an antigen and an antibody that specifically binds said antigen, comprising:
constructing a cell-free barcoded antigen display library comprising a plurality of plasmids encoding a plurality of antigens and a plurality of antigen barcodes, wherein each plasmid comprises a nucleic acid sequence encoding an antigen and a unique antigen barcode;
generating an antigen-barcode dictionary by mapping each unique antigen barcode to its corresponding antigen;
performing in vitro transcription of each plasmid to produce an mRNA transcript, wherein the mRNA transcript encodes the antigen and the unique antigen barcode;
reverse transcribing the mRNA transcript encoding the unique antigen barcode to form a corresponding cDNA;
performing in vitro translation of the mRNA transcript to express a cell-free barcoded antigen;
allowing a plurality of cell-free barcoded antigens to bind to a population of B-cells;
washing unbound cell-free barcoded antigens from the population of B-cells;
separating the B-cells bound to the cell-free barcoded antigens into single cell emulsions;
introducing a unique cell barcode-labeled bead into each single cell emulsion;
preparing a single cell cDNA library from each single cell emulsion, wherein the cDNA library comprises nucleic acid sequences encoding immunoglobulin heavy chain and/or immunoglobulin light chain sequences and the unique antigen barcode;
performing PCR amplification reactions to generate a plurality of amplicons comprising: (1) the unique cell barcode, a unique molecular identifier (UMI) and the unique antigen barcode, (2) the unique cell barcode, the immunoglobulin heavy chain and/or immunoglobulin light chain sequences, and the unique molecular identifier (UMI);
sequencing the plurality of amplicons and removing sequences lacking any of the unique cell barcode, the UMI, the unique antigen barcode, or the immunoglobulin sequence;
aligning the immunoglobulin sequences to a reference library of V, D, J, and C gene segments to annotate antibody sequences;
constructing a UMI count matrix comprising the unique cell barcode, the unique antigen barcode, and the corresponding antibody sequence; and
determining a LIBRA-seq score for each antigen-antibody pair based on the UMI count matrix.

2. The method of claim 1, wherein the plasmid further comprises a T7 promoter, a ribosome binding site (RBS), an N-terminal Tag, and an epitope tag.

3. The method of claim 2, wherein the N-terminal Tag comprises HaloTag, HA Tag or HIS Tag.

4. The method of claim 3, wherein the N-terminal Tag is a HaloTag.

5. The method of claim 1, wherein the unique antigen barcode is reverse transcribed using a primer comprising a HaloLigand moiety, wherein the HaloLigand moiety covalently links to N-terminal HaloTag of translated antigens.

6. The method of claim 1, wherein the cell-free barcoded antigens are not purified prior to incubation with the population of B-cells.

7. The method of claim 2, wherein the epitope tag is a FLAG tag, wherein the B-cells bound to the cell-free barcoded antigens are isolated using an antibody against the epitope tag.

8. The method of claim 1, wherein the barcode comprises a degenerate at least 10-nucleotide long sequence synthesized from a randomized oligonucleotide pool.

9. The method of claim 1, wherein the cell-free barcoded antigens comprise an antigen from a pathogen or an animal.

10. The method of claim 9, wherein the antigen from the animal comprises a tumor-associated antigen or a neoantigen.

11. The method of claim 9, wherein the antigen from a pathogen comprises an antigen from a nosocomial infection causing bacteria.

12. The method of claim 11, wherein the nosocomial infection causing bacteria comprises Staphylococcus aureus, Acinetobacter baumannii, Clostridioides difficile, or a combination thereof.

13. The method of claim 9, wherein the antigen from a pathogen comprises an antigen from a virus.

14. The method of claim 13, wherein the virus comprises HIV-1, SARS-CoV-2, SARS-CoV-1 or MERS.

15. A method for simultaneous detection of a host receptor protein and a neutralizing antibody that blocks the interaction of said host receptor protein with an antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a receptor protein-barcode dictionary by mapping each unique receptor protein barcode to its corresponding host receptor protein;
expressing the plurality of plasmids in a cell-free system; thereby obtaining barcoded host receptor proteins;
constructing a barcoded antigen library comprising a plurality of plasmids encoding a plurality of antigens;
expressing the plurality of antigens in a cell culture; wherein each antigen is associated with a unique antigen barcode, thereby obtaining barcoded antigens;
contacting B cells to the barcoded antigens and a barcoded host receptor protein-barcoded antigen complex;
sorting B cells bound to the barcoded antigens and/or the barcoded host receptor protein-barcoded antigen complex;
partitioning the sorted B cells into single-cell emulsions;
introducing a unique cell barcode-labeled bead into each single-cell emulsion;
synthesizing cDNA from each sorted B cell;
amplifying immunoglobulin variable region sequences (V(D)J) sequences, from each B cell with the unique antigen barcode and the unique receptor protein barcode, wherein the V(D)J sequences encode the antigen-binding regions of B cell receptors (BCRs) or secreted antibodies;
performing PCR amplification reactions to generate a plurality of amplicons comprising: (1) the unique cell barcode, a unique molecular identifier (UMI), and the unique antigen barcode, and (2) the unique cell barcode, the unique receptor protein barcode, and the unique molecular identifier (UMI);
sequencing the plurality of amplicons and removing sequences lacking any of the unique cell barcode, the UMI, the unique antigen barcode, or the unique receptor protein barcode;
constructing a UMI count matrix comprising the unique cell barcode, the unique receptor protein barcode, and the unique antigen barcode;
calculating a LIBRA-seq score for each combination comprising the unique cell barcode, the unique receptor protein barcode, and the unique antigen barcode based on the UMI count matrix; and
identifying the neutralizing antibody that blocks the interaction of the host receptor protein with an antigen.

16. A method for simultaneous detection of an antigen and a host receptor protein that specifically binds said antigen, comprising:
constructing a cell-free barcoded host receptor protein display library comprising a plurality of plasmids encoding a plurality of host receptor proteins, wherein each plasmid comprises a nucleic acid sequence for a host receptor protein and a unique receptor protein barcode;
generating a protein-barcode dictionary by mapping each unique receptor protein barcode to its corresponding host receptor protein;
performing in vitro transcription of each plasmid to produce an mRNA transcript; wherein the mRNA transcript encodes the host receptor protein and the unique receptor protein barcode;
reverse transcribing the mRNA transcript encoding the unique receptor protein barcode to form a corresponding cDNA;
performing in vitro translation of the mRNA transcript to express a cell-free barcoded host receptor protein;
contacting a plurality of cell-free barcoded host receptor proteins with a yeast display library expressing a plurality of antigens, wherein each antigen is attached to a unique antigen barcode;
allowing the plurality of cell-free barcoded host receptor proteins to bind to the plurality of antigens of the yeast display library to form a receptor-antigen binding complex comprising cell-free barcoded host receptor proteins bound to yeast cells;
washing unbound cell-free barcoded host receptor proteins from antigen expressing yeast cells;
separating the antigen expressing yeast cells bound to the cell-free barcoded host receptor proteins into single cell emulsions;
introducing a unique cell barcode-labeled bead into each single cell emulsion;
preparing a single cell cDNA library from each single cell emulsion, wherein the cDNA library comprises nucleic acid sequences encoding the unique antigen barcode and the unique receptor protein barcode;
performing PCR amplification reactions to generate a plurality of amplicons comprising: (1) the unique cell barcode, a unique molecular identifier (UMI) and the unique antigen barcode, and (2) the unique cell barcode, the unique receptor protein barcode, and the unique molecular identifier (UMI);
sequencing the plurality of amplicons and removing sequences lacking any of the unique cell barcode, the UMI, the unique antigen barcode, or the unique receptor protein barcode;
constructing a UMI count matrix comprising the unique cell barcode, the unique receptor protein barcode, and the unique antigen barcode; and
determining a LIBRA-seq score for each antigen-receptor pair based on the UMI count matrix.

17. The method of claim 16, wherein the yeast display library is prepared by the following method:
preparing a plurality of yeast display vectors encoding a plurality of antigens, wherein each yeast display vector comprises a nucleic acid sequence for an antigen and a unique antigen barcode;
generating an antigen-barcode dictionary by mapping each unique antigen barcode to its corresponding antigen; and
transforming the yeast display vectors into Saccharomyces cerevisiae cells, wherein the S. cerevisiae cells induce surface expression of the yeast display vectors, thereby obtaining the yeast display library expressing the plurality of antigens with the unique antigen barcodes.

18. The method of claim 16, wherein the cell-free barcoded host receptor proteins comprise a receptor protein associated with viral infection.

19. The method of claim 18, wherein the receptor proteins comprise human receptor proteins.

20. The method of claim 19, wherein the human receptor proteins comprise proteins from human epithelial cells.


SEQUENCE LISTING (11 sequences)

SEQ ID NO: 1 - DNA, 141 bp, synthetic construct
  source 1..141: mol_type = other DNA
  misc_feature 1..141: note = UCI for cell-free display vector (Nucleotide sequence)
  misc_feature 1..6: note = restriction site SacI
  misc_feature 7..19: note = Complement of 10x TSO oligo
  misc_feature 20..59: note = Barcode; wherein S=G/C and W=A/T
  misc_feature 60..83: note = complement of truncated TruSeq small RNA read 1 sequence
  misc_feature 84..135: note = sequence added to increase distance of RT primer from RBS to 32 nt
  misc_feature 136..141: note = restriction site SpeI
gagctctttcttatatgggswswswswswswswswswaggaswswswswswswswswswtggaattctcgggtgccaaggaacggagatcgatcgatacggcttacctggagttcagacgtgtgctccgccggccactagt

SEQ ID NO: 2 - DNA, 176 bp, synthetic construct
  source 1..176: mol_type = other DNA
  misc_feature 1..22: note = Additional sequences for restriction enzyme digestion
  misc_feature 1..176: note = UCI for PCR and cloning (Nucleotide sequence)
  misc_feature 23..28: note = Restriction site: SacI
  misc_feature 29..41: note = Complement of 10x TSO oligo
  misc_feature 42..59: note = Barcode; wherein S=G/C and W=A/T
  misc_feature 64..81: note = Barcode; wherein S=G/C and W=A/T
  misc_feature 82..105: note = complement of truncated TruSeq small RNA read 1 sequence
  misc_feature 106..157: note = sequence added to increase distance of RT primer from RBS to 32 nt
  misc_feature 158..163: note = Restriction site: SpeI
  misc_feature 164..176: note = Additional sequences for restriction enzyme digestion
cgactcactatagggagaccacgagctctttcttatatgggswswswswswswswswswaggaswswswswswswswswswtggaattctcgggtgccaaggaacggagatcgatcgatacggcttacctggagttcagacgtgtgctccgccggccactagtaattttgtttaac

SEQ ID NO: 3 - DNA, 25 bp, synthetic construct
  source 1..25: mol_type = other DNA
  misc_feature 1..25: note = UCI Fwd primer for PCR amplification of the UCI oligos (Nucleotide sequence)
cgactcactatagggagaccacgag

SEQ ID NO: 4 - DNA, 27 bp, synthetic construct
  source 1..27: mol_type = other DNA
  misc_feature 1..27: note = UCI Rev primer for PCR amplification of the UCI oligos(Nucleotide sequence)
gttaaacaaaattactagtggccggcg

SEQ ID NO: 5 - DNA, 46 bp, synthetic construct
  source 1..46: mol_type = other DNA
  misc_feature 1..46: note = Fwd primer for reverse transcription (Nucleotide sequence)
ctcgggtgccaaggaacggagatcgatcgatacggcttacctggag

SEQ ID NO: 6 - DNA, 46 bp, synthetic construct
  source 1..46: mol_type = other DNA
  misc_feature 1..46: note = Rev primer for reverse transcription (Nucleotide sequence)
ctccaggtaagccgtatcgatcgatctccgttccttggcacccgag

SEQ ID NO: 7 - DNA, 177 bp, synthetic construct
  source 1..177: mol_type = other DNA
  misc_feature 1..177: note = UCI FOR YEAST SURFACE DISPLAY VECTOR (Nucleotide sequence)
  misc_feature 1..22: note = Additional sequences for restriction enzyme digestion
  misc_feature 23..28: note = Restriction site: SpeI
  misc_feature 29..41: note = Complement of 10x TSO oligo
  misc_feature 42..59: note = Barcode; wherein S=G/C and W=A/T
  misc_feature 64..81: note = Barcode; wherein S=G/C and W=A/T
  misc_feature 82..105: note = complement of truncated TruSeq small RNA read 1 sequence
  misc_feature 106..157: note = sequence added to increase distance of RT primer from RBS to 32 nt
  misc_feature 158..164: note = Restriction site: BlpI
  misc_feature 165..177: note = Additional sequences for restriction enzyme digestion
cgactcactatagggagaccacactagttttcttatatgggswswswswswswswswswaggaswswswswswswswswswtggaattctcgggtgccaaggaacggagatcgatcgatacggcttacctggagttcagacgtgtgctccgccggccgctgagcaattttgtttaac

SEQ ID NO: 8 - DNA, 27 bp, synthetic construct
  source 1..27: mol_type = other DNA
  misc_feature 1..27: note = UCI Fwd primer_pETcon (Nucleotide sequence)
cgactcactatagggagaccacactag

SEQ ID NO: 9 - DNA, 27 bp, synthetic construct
  source 1..27: mol_type = other DNA
  misc_feature 1..27: note = UCI Rev primer_pETcon (Nucleotide sequence)
gttaaacaaaattgctcagcggccggc

SEQ ID NO: 10 - DNA, 7423 bp, synthetic construct
  source 1..7423: mol_type = other DNA
  misc_feature 1..7423: note = Custom cell-free display vector with UCI/barcode (Nucleotide sequence)
  misc_feature 71..88: note = Barcode; wherein S=G/C and W=A/T
  misc_feature 93..110: note = Barcode; wherein S=G/C and W=A/T
atcgagatctcgatcccgcgaaattaatacgactcactatagggagaccacgagctctttcttatatgggswswswswswswswswswaggaswswswswswswswswswtggaattctcgggtgccaaggaacggagatcgatcgatacggcttacctggagttcagacgtgtgctccgccggccactagtcctatacaaataattttgtttaactttaagaaggagatataaccatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagcttattatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctatctacgctagaaatttccggcgactacaaagacgatgacgacaagtcggatctggttccgcgtccatggtcgaatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcaggcggggcgtaatctagaggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtttgattcgacccgggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagctagggataacagggtaatagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagagg

SEQ ID NO: 11 - DNA, 7924 bp, synthetic construct
  source 1..7924: mol_type = other DNA
  misc_feature 1..7924: note = Custom yeast display library vector with UCI/barcode (Nucleotide sequence)
  misc_feature 805..822: note = Barcode; wherein S=G/C and W=A/T
  misc_feature 827..844: note = Barcode; wherein S=G/C and W=A/T
attgtgagcggataacaatttcacacaggaaacagctatgaccatgattacgccaagctcgaaattaaccctcactaaagggaacaaaagctggtaccaattccttgaattttcaaaaattcttactttttttttggatggacgcaaagaagtttaataatcatattacatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcgaattcgagaccacactagttttcttatatgggwswswswswswswswswsaggawswswswswswswswswstggaattctcgggtgccaaggaacggagatcgatcgatacggcttacctggagttcagacgtgtgctccgccggccgctgagccctacttcatacattttcaattaagatgcagttacttcgctgtttttcaatattttctgttattgcttcagttttagcacaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgtttttaaggacaatagctcgacgattgaaggtagatacccatacgacgttccagactacgctctgcagacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcaggcggggcgtaatctagaggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggcatgcgtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtcagccggtcgaccatagtgactggatatgttgtgttttacagtattatgtagtctgttttttatgcaaaatctaatttaatatattgatatttatatcattttacgtttctcgttcagctttcttgtacaaagtggtggatccgaacaaaagcttatttctgaagaggacttgtaatagagatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcctttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtgga